SEQUENCE LISTING (21 sequences)

SEQ ID NO: 1 - DNA, 1449 bp, Danio rerio
atggacgagtctgatgggaagctgttccttactgaggctttcaacataatcatggaagaaattcttaacaaaggaagggacttgaaggagaaggtttgtgagtggaaagatccagatcagctgagatctctcctggacctcgaacttcgggatcatggagaatgtcatgagaagctgctgcagagggttcgagatgtggccaaatacagcgtaaaaacttgtcatcctcggttcttcaatcagctgtttgctggcgtggactatcatgcactgacaggacggctcatcactgaaaccctcaataccagccaatacacctatgaagtggctccagtgtttgtcctgatggaggaggaagtgatcagtaagcttcgctctctggttggctggtcagaaggagatgggatcttttgtcctggaggatccatgtctaacatgtatgccattaacgtcgctcggtactgggcttttcctcaagtgaagacaaaaggcttgtgggccgcaccacggatggctatatttacatcacaacagagtcattactccgtgaaaaaaggagctgcgtttcttggtattggaacagaaaatgttttcattgtgcaagtggatgagagcggcagcatgataccagaagacctggaggcaaaaattgtgcaggcaaaatcccaagacgctgttccgtttttcgtaaacgccacagccggaaccacagtgcagggagcctttgaccctctgaagcgcatagctgacatatgtgaaagaaacggcatgtggatgcatgttgacgccgcatggggaggaagcgtgctgttttccaaaaagcacagacatctggttgcaggaatagaaagagcaaactcggtgacttggaatcctcacaaaatgcttctgacgggactgcagtgctctgtgattttgttcagagatactacgaatttgctcatgcactgtcacagtgccaaagccacatacttgttccagcaagacaagttctacgacacaagtctggacacgggcgacaaatccatccagtgtggccggaaggtggattgcctcaagctctggctcatgtggaaggcaatcggagctagtggtctttcacagcgtgtcgataaggcctttgccctcactaggtatttagttgaagaaatggagaaacgggagaatttccagctggtctgtaaggggccgtttgtgaacgtttgcttctggtttattccacccagtctgaaaggaaaggagaacagcccagattaccaggaaagactatccaaggtggcgccagtcattaaagagaggatgatgaagcgaggaacgatgatggtgggatatcagccaatggatgaacacgtcaacttcttccgcatggtggttgtttctccacagctcacaaccaaagacatggatttcttccttgatgagatggagaaactcgggaaggatctatga

SEQ ID NO: 2 - DNA, 1449 bp, Artificial Sequence
  Synthetic Construct
atggatgagtctgatggaaagttgttcctcactgaggctttcaacatcatcatggaagagatcctcaacaagggaagggatctcaaagaaaaggtgtgcgaatggaaagatcctgatcagttgagatctttgctcgatctcgagcttagagatcatggtgagtgtcatgagaagttgcttcagagggttagagatgtggctaagtactctgttaagacttgccaccctagattcttcaaccagctttttgctggtgtggattaccatgctcttactggtagactcatcactgagactctcaacacttctcagtatacctacgaagttgctcctgttttcgtgcttatggaagaagaggtgatcagtaagctcagatcacttgttggatggtctgagggtgatggaattttttgtcctggtggatctatgtctaacatgtacgctatcaacgtggcaagatattgggctttcccacaggttaagactaagggactttgggctgctcctagaatggctattttcacttctcagcagtctcactactctgtgaagaagggtgctgcttttcttggaatcggaactgagaacgtgttcatcgttcaagttgatgagtctggatctatgatccctgaagatcttgaggctaagatcgttcaagctaagtctcaagatgctgtgcctttcttcgttaatgctactgctggaactactgttcagggagcttttgatcctctcaagagaatcgctgatatctgtgagagaaacggaatgtggatgcatgttgatgctgcttggggaggatctgttctcttcagtaagaagcacagacatctcgttgctggaattgagagagctaactctgttacttggaaccctcataagatgcttctcactggacttcagtgctctgttatcctcttcagggatacaactaacctcctcatgcattgccattctgctaaggctacttaccttttccagcaggataagttctacgatacctctctcgataccggtgataagtctatccagtgcggaagaaaagtggattgtcttaagctctggctcatgtggaaggctattggagcttctggactttctcagagagtggataaggctttcgctcttactagatacctcgtggaagagatggaaaagcgtgagaacttccagcttgtgtgcaagggacctttcgttaacgtttgcttctggttcatccctccatctcttaagggaaaagagaactctcctgattaccaagagaggctctctaaagttgctccagtgatcaaagaacgtatgatgaagaggggtactatgatggttggataccaacctatggatgagcacgtgaactttttcagaatggttgttgtgtctcctcagctcactaccaaggatatggatttcttcttggacgaaatggaaaagctcggaaaggatctctga

SEQ ID NO: 3 - DNA, 1527 bp, Takifugu rubripes
atgtttttaaatgttttatcaacagcaattgggctggtagaggacctagccggtccccagcaccctgaagactccctcgttaatcactcggagggacagttcttcctcaacgaagccttcaaaattattgtcgacgaggtgctcaacaaaggcaccgacgtccagcaaaaggtttgtgattggaaagaaccagaggagctggctcagctgctggacctggagctgagagcagcgggcgagccacatcacaggctcctggagagagtccgggacgtggccaagtacagcatcaaaacaagtcatccgcggtttttcaatcagcagtttgctggggtggactaccattccctggctgggagattcctctctgaggctctcaacacgaacctctacacctatgaggccgctccggtgtttgtgctgatggaaaacgaggtgctgagaagtttgcgtctgctgattggttggacggagggcgatggcattttctgcccgggggggtccacctccaacatgtacgccatgaatctcgcacgcttccagctcttccccgaggtcaaaagccaggggctgtgcgggcttcctcgacttgccatctttacatcttcgcagagccattattctgtgaagaaaggggcgggctttctgggcatcggaacagacaatgtcatcttggtgaaagtggacgatggaggctgcatgattccagaggacctggatgaaaagatagcgctggtaaaatctcagggcacggtgccttttattgtcagctgcacagcaggaaccacagtgcagggtgccttcgaccctctggaccgtatagctgatgtctgtgagaagcacaagctctggatgcatgtggacggtgcctggggagcgagtgttctcttctccaagcaacacagacatctgttgaaaggcatagaaagagcaaattcagttgcttggaacccacataagctgctgctggctggtcttcagtgctctgccctgctgttgcgggactgcacgggtttgctgaagcggtgccacgttgccaatgctacatacctgttccagcaagacaaattctatgatctcgatctggatattgggaacaagtcgctgcagtgcagccgtaaggttgactgtctcaaactgtggctcatgtggaaagctgttggctctagtggcttggaagaacgtgtgaataaagcatttttcaacgcaaggtactttgtacaggagctgaagaaaagaaaaggcttttatcttctgcatgagccagagtttttgaatatctgcttctggttcataccgcccagtctgcggggaaaggaaggaaatccagattaccaggaaaaactggccaaagtggctccagccatcaaagagcgcatgatgaaaaaaggcagcatgatggtcggataccaacctttagaaaacaaagtcaactttttccgcatggttgtcctgtctccactggtttcccaggaagacatggatttctgtctggatgaaattgaaaggcttggtaatgacatgtga

SEQ ID NO: 4 - DNA, 1503 bp, Cyprinus carpio
atgagctcatctaaagaatatatgaatggccatgtccacctggaagaatcagacatgtatgagtcagatggcaagctgttccttactgaggctttcaatgtgattatggaggagatacttcacaaaggaactgactcggaggagaaggtgtgtgagtggaaagatcccgatcagctgagagctcttctggaccttgatctcagagagcatggagaatctcacaagcagttactgcagagggttcgagatgtggccagatacagtgttaaaacctgtcatcctcggttcttcaatcagctgtttgctggcgtggactaccatgcactgacaggacggctcctcactgaaaccctcaacaccagccaatacacctatgaagtggctccggtgtttgtcctgatggaggaggaagtgctctgtaagcttcgctctctggttggctggtcagaaggagacgggatattttgtcctgggggttcaatgtccaacatgtatgccatgaacgtcgcacggtactgggccttccctcaagtgaagacacaaggcttgtgggccaccccacgaatggctatatttacatcacaacagggtcattactcaatgaaaaaagcagctgctttccttggtatcggaacggacaacgttttcattgtacaagtggatgagagtggcagcatgagaccagaggacctagaggcgaaaatcgtgcaggcaaaatcacaagatgctgtcccgtttttcgttaatgccacagcgggtaccacagtgcagggcgcctttgatcctctgaaccgcatagctgacataagtgaaagaaacggcatgtggatgcacgttgacgccgcttggggaggaagcgtgctgttttccaaaaaacataaacatctggttgcaggaatagaaagagcaaactctgtgacttggaaccctcacaaaatgcttctagcgggattgcagtgctctgtggttttgttcagagataccacgaatctgttaatgcactgtcacagtgccaaggcaacctacttattccagcaagacaagttctacgatacaagtctagacacgggagacaaatccatccagtgtggccgcaaggtggattgtttgaagctgtggttgatgtggaaggcaataggaacacatggtctttcagagcgagtggagaaggcctttgccctcgcaaggtatttagttgaagaaatggagaaaagggacaatttcaagttggtctgtaaggggccttttgtgaacgtttgcttctggttcattccacccagtctgagaaggaaggagaacagtgcagattaccaggaaaggttatcaaaggtggcgccggtcatcaaagagagaatgatgaagcgtggaaccatgatggtgggataccagcccatggatggacacgtcaacttcttccgtatggttgttgtttctcctcagctgaccaccaaggacatggatttcttccttgatgagatagaggacctagggaaggacttgtag

SEQ ID NO: 5 - DNA, 1482 bp, Bos taurus
atggctgactctcaacctctcctctcccttgatggggaccctgtggctgcagaagccttgctccaggacgtgtttgggattgtggtggatgaggtcattcgaaaagggaccagtgcctccgagaaggtctgtgagtggaaggagcccgaggagctgaagcaactgctggacttggagctgcggcacgagggcgagtcacaggagcagatcctggagcactgccgggccgtgatccgctacagcgtgaagacctgtcaccctcgtttcttcaaccagctcttctcagggttggatccccatgccctggccgggcgcattgtcacagagagcctcaacaccagccagtacacgtacgaaatcgcccctgtgtttgtcctcatggaggaggaggtgctgaagaaactccgggccctggtgggctggagctctggggatggggtcttctgccctggtggctccatctccaacatgtatgcagtgaacctggcccgctatcagcgatacccagactgcaaacagaggggcctccgggcactgccgcccctggccctcttcacatcgaaagagtgtcattactccatcaagaaaggagccgcttttctgggacttggcaccgacagtgtccgagtggtcaaggcagatgagagagggaaaatgatccctgaggatctggagaggcagatcagcttggctaaggccgagggtgccgtgccattcctggtcagtgccacctctggtactacagtgctgggggcctttgaccccctggaggcaatcgcagatgtgtgccagcatcacgggctgtggctgcatgtggacgctgcctggggtggaagcgtcctgctgtcacagacacatagacatctcctggctgggatccagagggctgactccgtggcctggaatccccacaagctcctctccacaggcctgcagtgctcagctcttcttctccgggacacctcgaacctgctcaagcgctgtcacgggtcccaggccagctacctcttccagcaagacaagttctacgatgtggctctggacacaggagacaaggtggtgcagtgtggccggcgtgtggactgtctgaagctgtggctcatgtggaaggcacagggcgagcaggggctgcagcgccgtgtggaccaggcctttgcccttgcccggtacctggtggaggagctgaagaagcgggagggatttgagttggtcatggagcctgaatttgtcaatgtgtgtttctggttcgtgccccccagtctgcgcgggaagaaggggagtccagattacagtgaaaggctgtctaaggtagccccaatcctcaaggagcgcatggtgaggaagggttccatgatgattggctaccagccccatggtacccggagcaacttctttcgcatggttgtggccaaccctgcactgacacgggctgatatggacttcctgctgaacgagctggaacggctgggccaggatctctga

SEQ ID NO: 6 - DNA, 1545 bp, Sus scrofa
atgcgaataactcctgctctgctcacagaccttatcctgatggctgactccaaaccactcctctcccttgatggggaccccgcggctgcagaagctttgctccaggatgtgtttggcatcgtggtggatgaggtcattaggaaagggaccagcgcctccgagaaggtctgcgagtggaaggagccagaggagctgaaacagctgctggacttggagctgcgcagcgagggggagtcacaggagcagatcctagagcggtgccgggctgtgatccgctacagcgtgaagacctgtgggtcctgggtcccggagggaggatcaccctcgtttcttcaccagctcttctcaggtctctcccccactcctctaagagtccctccagttctcattatcccctctcctggcaggtacacgtatgaaatcgcccccgtgtttgtactcatggaagaagaggtgctgaagaaactccgggccctggtgggctggagctctggggatggggtcttttgccctggtggctccatctccaacatgtatgctatgaacctggcccgctatcagcgctacccggattgcaaacagaggggcctccgggcactgccacccctggccctttttacatccaaggagtgtcattactccatcaagaagggagctgcctttctgggacttggcaccgacagtgtccgaatagtccaggcagatgagagagggaaaatgatccctgaggatctggagcagcagatcagactggctgaggctgagggtgctgtgccatttctggtcagtgccacctctggcactaccgtgctgggggcctttgaccccctggaggccattgctgatgtgtgccagcgtcacgggctgtggctgcacgtggatgccgcctggggggggagtgttctgctgtcacagacccatcgacatcttctggatgggatccagagggctgactccgtggcctggaatccccacaagctcctctccgtaggcctgcagtgttcggctctccttctccgagacacgtcgaacctgctcaagcgctgccacgggtcccaggccagctacctcttccagcaggacaagttctacgacgtgaccctggacacgggggacaaggtggtgcagtgtggccgccgcgtggactgtctgaagctgtggctcatgtggaaggcacagggcgggcaggggctggagcggcgagtggaccaggcctttgcccttgcccggtacctggcggaggaattgaagaagcgggaggggtttgagttggtcatggagcctgaatttgtcaatgtgtgtttctggttcgtgccccccagtctgcggggaaagaaggggagtccggattacaatgaaaggctggctaaggtggcgcccatcctcaaggagagcatggtgaagaagggctccatgatgatcggctaccagccccacggcacccggggcaacttcttccgcatggtcgtggccaaccctgcactgacccgggttgatatggacttcctgctgaacgagctggaacagctgggccaggacctctga

SEQ ID NO: 7 - DNA, 1593 bp, Xenopus (Silurana) tropicalis
atggagcttatatgttctggttatgtggttgttcagctgattgtgattagctcagtgcccgccatattaaacacatcagatttactttcttggcacagtacctctgctgaaatagatttatggcgctgtatatatcaccctgtagtggacagtggaagtggagatgaattcttgaaagaagctcttgttattttaatgcaggatgcagtgcaaaaggggacagacaggagccacaaggtgtgtgagtggatggaaccagagcaacttcatgaaatcctgaacttaaaactacagagacacggcgaatctcagcaagcattgctgggctactgcagagacattgtcaggtttagtgtcaagacagggcatccacgcttcttcaaccagctgttttctggattagaccgacatgcgctgactggtcgcattctgagcgagaccttgaacactagtcaatatacctatgaggttgctccagtttttgttttaatggaagaagaagttctcagaactctgaggcattatgttggctggacggctggagatgggattttctgcccaggaggctctatttccaacatgtatgctataaatgtggcccgatttcagcgcttcccacatgctaaagagaaaggattgagttccctgccaaggatgactatattcacaacacaagagagccattattcagtcagaaagggtgcagcattcatgggcattggaactgacaacatcatcattgtacagacaaatgacagagggaaaatgttgcctgaagatttagagaaccaaattcagaaatcaagatcggagggttctgtgcctttcttggtcagcgcaacttgcggcacaactgttctaggtgcttttgatcctttagcagaaattgctgatgtatgtgaacagcatggattatggctgcatgtggatgctgcttttggtggcagtgccctcttgtccaacaagcacaggcaccttttaaatggcatagaaagggcaaattctgtcacttggaatcctcacaagatgctgggtgtaggtttgcaatgttcagcctttctccttccggacacgacaggactattacagcgatgccatgcagcttatgccacatatctcttccaaacggacaaattttatgaccttcagtatgacactggagacaaatcaatccagtgtggtcgaagggtggattgcttaaagctttggctgatgtggaaggcacttgggtctgaaggtttggaaaagcgagtggacaaggcattggaacaaaccagatatttgatggaagagatgacgaagagggaaggtttttgtctaattatggagccagaatttgtgaacctgtgtttctggtatgtgccacctagccttcgacaccagcagaattcccctgattactgggagagactaggaaaggtagctccagtaataaaagagagaatgatgaagaaaggttctatgatggttgggtaccagccccatggaaacagagtaaacttctttcgtcaagttgtagtgaatcctgcagtaactaaaagagacctggattttttcctggatgaaatagaactattagcagaggacctctga

SEQ ID NO: 8 - protein, 482 aa, Danio rerio
MDESDGKLFLTEAFNIIMEEILNKGRDLKEKVCEWKDPDQLRSLLDLELRDHGECHEKLLQRVRDVAKYSVKTCHPRFFNQLFAGVDYHALTGRLITETLNTSQYTYEVAPVFVLMEEEVISKLRSLVGWSEGDGIFCPGGSMSNMYAINVARYWAFPQVKTKGLWAAPRMAIFTSQQSHYSVKKGAAFLGIGTENVFIVQVDESGSMIPEDLEAKIVQAKSQDAVPFFVNATAGTTVQGAFDPLKRIADICERNGMWMHVDAAWGGSVLFSKKHRHLVAGIERANSVTWNPHKMLLTGLQCSVILFRDTTNLLMHCHSAKATYLFQQDKFYDTSLDTGDKSIQCGRKVDCLKLWLMWKAIGASGLSQRVDKAFALTRYLVEEMEKRENFQLVCKGPFVNVCFWFIPPSLKGKENSPDYQERLSKVAPVIKERMMKRGTMMVGYQPMDEHVNFFRMVVVSPQLTTKDMDFFLDEMEKLGKDL

SEQ ID NO: 9 - protein, 508 aa, Takifugu rubripes
MFLNVLSTAIGLVEDLAGPQHPEDSLVNHSEGQFFLNEAFKIIVDEVLNKGTDVQQKVCDWKEPEELAQLLDLELRAAGEPHHRLLERVRDVAKYSIKTSHPRFFNQQFAGVDYHSLAGRFLSEALNTNLYTYEAAPVFVLMENEVLRSLRLLIGWTEGDGIFCPGGSTSNMYAMNLARFQLFPEVKSQGLCGLPRLAIFTSSQSHYSVKKGAGFLGIGTDNVILVKVDDGGCMIPEDLDEKIALVKSQGTVPFIVSCTAGTTVQGAFDPLDRIADVCEKHKLWMHVDGAWGASVLFSKQHRHLLKGIERANSVAWNPHKLLLAGLQCSALLLRDCTGLLKRCHVANATYLFQQDKFYDLDLDIGNKSLQCSRKVDCLKLWLMWKAVGSSGLEERVNKAFFNARYFVQELKKRKGFYLLHEPEFLNICFWFIPPSLRGKEGNPDYQEKLAKVAPAIKERMMKKGSMMVGYQPLENKVNFFRMVVLSPLVSQEDMDFCLDEIERLGNDM

SEQ ID NO: 10 - protein, 500 aa, Cyprinus carpio
MSSSKEYMNGHVHLEESDMYESDGKLFLTEAFNVIMEEILHKGTDSEEKVCEWKDPDQLRALLDLDLREHGESHKQLLQRVRDVARYSVKTCHPRFFNQLFAGVDYHALTGRLLTETLNTSQYTYEVAPVFVLMEEEVLCKLRSLVGWSEGDGIFCPGGSMSNMYAMNVARYWAFPQVKTQGLWATPRMAIFTSQQGHYSMKKAAAFLGIGTDNVFIVQVDESGSMRPEDLEAKIVQAKSQDAVPFFVNATAGTTVQGAFDPLNRIADISERNGMWMHVDAAWGGSVLFSKKHKHLVAGIERANSVTWNPHKMLLAGLQCSVVLFRDTTNLLMHCHSAKATYLFQQDKFYDTSLDTGDKSIQCGRKVDCLKLWLMWKAIGTHGLSERVEKAFALARYLVEEMEKRDNFKLVCKGPFVNVCFWFIPPSLRRKENSADYQERLSKVAPVIKERMMKRGTMMVGYQPMDGHVNFFRMVVVSPQLTTKDMDFFLDEIEDLGKDL

SEQ ID NO: 11 - protein, 493 aa, Bos taurus
MADSQPLLSLDGDPVAAEALLQDVFGIVVDEVIRKGTSASEKVCEWKEPEELKQLLDLELRHEGESQEQILEHCRAVIRYSVKTCHPRFFNQLFSGLDPHALAGRIVTESLNTSQYTYEIAPVFVLMEEEVLKKLRALVGWSSGDGVFCPGGSISNMYAVNLARYQRYPDCKQRGLRALPPLALFTSKECHYSIKKGAAFLGLGTDSVRVVKADERGKMIPEDLERQISLAKAEGAVPFLVSATSGTTVLGAFDPLEAIADVCQHHGLWLHVDAAWGGSVLLSQTHRHLLAGIQRADSVAWNPHKLLSTGLQCSALLLRDTSNLLKRCHGSQASYLFQQDKFYDVALDTGDKVVQCGRRVDCLKLWLMWKAQGEQGLQRRVDQAFALARYLVEELKKREGFELVMEPEFVNVCFWFVPPSLRGKKGSPDYSERLSKVAPILKERMVRKGSMMIGYQPHGTRSNFFRMVVANPALTRADMDFLLNELERLGQDL

SEQ ID NO: 12 - protein, 514 aa, Sus scrofa
MRITPALLTDLILMADSKPLLSLDGDPAAAEALLQDVFGIVVDEVIRKGTSASEKVCEWKEPEELKQLLDLELRSEGESQEQILERCRAVIRYSVKTCGSWVPEGGSPSFLHQLFSGLSPTPLRVPPVLIIPSPGRYTYEIAPVFVLMEEEVLKKLRALVGWSSGDGVFCPGGSISNMYAMNLARYQRYPDCKQRGLRALPPLALFTSKECHYSIKKGAAFLGLGTDSVRIVQADERGKMIPEDLEQQIRLAEAEGAVPFLVSATSGTTVLGAFDPLEAIADVCQRHGLWLHVDAAWGGSVLLSQTHRHLLDGIQRADSVAWNPHKLLSVGLQCSALLLRDTSNLLKRCHGSQASYLFQQDKFYDVTLDTGDKVVQCGRRVDCLKLWLMWKAQGGQGLERRVDQAFALARYLAEELKKREGFELVMEPEFVNVCFWFVPPSLRGKKGSPDYNERLAKVAPILKESMVKKGSMMIGYQPHGTRGNFFRMVVANPALTRVDMDFLLNELEQLGQDL

SEQ ID NO: 13 - protein, 530 aa, Xenopus (Silurana) tropicalis
MELICSGYVVVQLIVISSVPAILNTSDLLSWHSTSAEIDLWRCIYHPVVDSGSGDEFLKEALVILMQDAVQKGTDRSHKVCEWMEPEQLHEILNLKLQRHGESQQALLGYCRDIVRFSVKTGHPRFFNQLFSGLDRHALTGRILSETLNTSQYTYEVAPVFVLMEEEVLRTLRHYVGWTAGDGIFCPGGSISNMYAINVARFQRFPHAKEKGLSSLPRMTIFTTQESHYSVRKGAAFMGIGTDNIIIVQTNDRGKMLPEDLENQIQKSRSEGSVPFLVSATCGTTVLGAFDPLAEIADVCEQHGLWLHVDAAFGGSALLSNKHRHLLNGIERANSVTWNPHKMLGVGLQCSAFLLPDTTGLLQRCHAAYATYLFQTDKFYDLQYDTGDKSIQCGRRVDCLKLWLMWKALGSEGLEKRVDKALEQTRYLMEEMTKREGFCLIMEPEFVNLCFWYVPPSLRHQQNSPDYWERLGKVAPVIKERMMKKGSMMVGYQPHGNRVNFFRQVVVNPAVTKRDLDFFLDEIELLAEDL

SEQ ID NO: 14 - DNA, 21 bp, Artificial Sequence
  Primer
caatgcctaataatgtctagc

SEQ ID NO: 15 - DNA, 46 bp, Artificial Sequence
  Primer
cagcttcccatcagactcgtccatgccgtttgattttgaatttgag

SEQ ID NO: 16 - DNA, 24 bp, Artificial Sequence
  Primer
atggacgagtctgatgggaagctg

SEQ ID NO: 17 - DNA, 22 bp, Artificial Sequence
  Primer
tcatagatccttcccgagtttc

SEQ ID NO: 18 - DNA, 42 bp, Artificial Sequence
  Primer
gaaactcgggaaggatctatgagctaccgagctcgaatttcc

SEQ ID NO: 19 - DNA, 22 bp, Artificial Sequence
  Primer
cacgacgttgtaaaacgacggc

SEQ ID NO: 20 - DNA, 32 bp, Artificial Sequence
  Primer
aaaaatctagaattcttgaattacgattgtac

SEQ ID NO: 21 - DNA, 35 bp, Artificial Sequence
  Primer
tttttttgtcgacgatctagtaacatagatgacac

(19) United States
(12) Patent Application Publication
(10) Pub. No.: US 2014/0082761 A1
(43) Pub. Date: Mar. 20, 2014

(54) METHODS TO INCREASE PLANT PRODUCTIVITY

(71) Applicants: Frank J. Turano, Baltimore, MD (US); Kathleen A. Turano, Baltimore, MD (US)

(72) Inventors: Frank J. Turano, Baltimore, MD (US); Kathleen A. Turano, Baltimore, MD (US)

(73) Assignee: PLANT SENSORY SYSTEMS, LLC, Baltimore, MD (US)

(21) Appl. No.: 14/115,943

(22) PCT Filed: Apr. 17, 2012
(86) PCT No.: PCT/US2012/033916

§ 371 Date: Nov. 6, 2013

Related U.S. Application Data
(60) Provisional application No. 61/487,412, filed on May 18, 2011.

(51) Int. Cl. C12N 15/82; A01H 5/10

(52) U.S. Cl. CPC C12N 15/8241; A01H 5/10
USPC 800/260; 424/757; 424/750; 424/725; 424/755; 435/419; 435/257.2; 800/298; 800/301; 800/302; 800/320; 800/313; 800/306; 800/309; 800/320.1; 800/314; 800/307; 800/316; 800/317; 800/323; 800/317.1; 800/319; 800/317.2; 800/310; 800/320.2; 800/312; 800/322; 800/317.3; 800/317.4; 800/320.3; 426/648

(57) ABSTRACT

The present invention describes an approach to increase plant growth and production. The invention describes methods for the use of functional sulfinoalanine decarboxylase (SAD) or the promiscuous enzyme activity of SAD in plants or algal cells. Transgenic plants will have increased plant growth, biomass, yield, and/or tolerance to biotic and/or abiotic stresses and could be used as a pharmaceutical, nutraceutical or as a supplement in animal feed.

### CROSS-REFERENCE TO RELATED APPLICATION

The present application is related to and claims priority to U.S. provisional patent application Ser. No. 61/487,412 filed 18 May 2011. This application is incorporated herein by reference in its entirety.

### SEQUENCE SUBMISSION

The present application is being filed along with a Sequence Listing in electronic format. The Sequence Listing is entitled 3834111PCTSequenceListing.txt, was created on 17 Apr. 2012 and is 42 kb in size. The information in the electronic format of the Sequence Listing is part of the present application and is incorporated herein by reference in its entirety.

### FIELD OF THE INVENTION

The present invention is in the field of genetic modification of plants.

### BACKGROUND OF THE INVENTION

The publications and other materials used herein to illuminate the background of the invention, and in particular, cases to provide additional details respecting the practice, are incorporated by reference in their entirety for all that they disclose, and for convenience are referenced in the following text by reference number and are listed by reference number in the appended bibliography.

Sulfinoalanine Decarboxylase (SAD)

SAD is a pyridoxal phosphate (PLP) dependent enzyme that catalyses the reaction for the conversion of 3-sulfino-L-alanine into 2-aminoethanesulfinic acid (hypotaurine) plus CO₂. In animals, SAD is one enzyme in a multi-step pathway for taurine (2-aminoethane sulfonate or 2-aminoethane sulfonic acid) biosynthesis. The SAD gene, SAD enzyme, and other components of the SAD-mediated taurine pathway do not exist in plants or bacteria (1).

SAD as a Promiscuous Enzyme

There are several reports (2-6) from in vitro assays on SAD isolated from rat that show that SAD is a promiscuous enzyme with lower affinities for several substrates such as aspartate, glutamate and cysteic acid. The affinity of an enzyme for a specific substrate is determined by the Michaelis-Menten constant (Kₘ). The lower the Kₘ the higher the affinity of the enzyme for the particular substrate. As shown in Table 1, the substrate with the lowest Kₘ, and hence highest affinity, for SAD is 3-sulfino-L-alanine, which is its preferred substrate. However in the absence, or at low concentrations, of its preferred substrate SAD will use other substrates and convert them into other end products through a decarboxylation reaction. SAD can convert aspartate into beta-alanine+CO₂, glutamate into 4-aminobutanoate+CO₂, and cysteic acid into 2-aminoethane sulfonate+CO₂ (FIG. 1).

TABLE 1
Various substrates and their Kₘ values for SAD.

| Substrate | Kₘ (mM) | End Products | Reference(s) |
|---|---|---|---|
| 3-sulfino-L-alanine | 0.033 to 2.0 | 2-aminoethanesulfinic | (2, 3, 4, 5, 6) |
| | | acid + CO₂ | |
| aspartate | 11.0 | beta-alanine + CO₂ | (5) |
| glutamate | 2.9 | 4-aminobutanoate + | (2) |
| | | CO₂ | |
| cysteic acid | 0.66 to 4.0 | 2-aminoethane | (2, 6) |
| | | sulfonate + CO₂ | |

Potential Benefits of Promiscuous Enzyme Activity of SAD

The end-products of the promiscuous enzyme activity of SAD (FIG. 1), namely beta-alanine, 4-aminobutanoate, and 2-aminoethane sulfonate, have been shown to promote plant growth and production. The agronomic benefits of these compounds have been demonstrated by the exogenous application to plants or by gene modification of plants. In addition, several of the end-products of the promiscuous enzyme activity of SAD could also enhance the nutritional quality of plants.

The non-protein amino acid, beta-alanine, could improve the nutritional quality of plant since it is a precursor for vitamin B5. In addition, in plants that are members of the Plumbaginaceae family beta-alanine can form beta-alanine betaine, an osmoprotectant. Plants that express a bacterial aspartate decarboxylase (panD) gene have increased levels of beta-alanine and pantothenic acid, a precursor to vitamin B5, and are more thermotolerant during germination than plants without the panD gene (7). In addition plants that express the panD gene have increased photosynthesis and biomass production under elevated temperature than plants without the panD gene (8).

The non-protein amino acid 4-aminobutanoate also known as gamma-aminobutyric acid (GABA) may confer multiple agronomic benefits to plants or crops, such as increased plant growth and development (9, 10); increased tolerance to abiotic stresses, including drought (11), salinity (12), flooding (13), heat (14), freezing (15, 16), limited nutrient availability (including limited nitrogen, phosphorous, potassium and other minerals) (17); and increased tolerance to biotic stresses, such as insect feeding (18, 19) and nematode infestation (160). GABA may function to alter nitrogen and/or carbon metabolism (20-24). GABA may also increase nitrogen use efficiency, nutrient use efficiency and water use efficiency in plants.

With very few exceptions (25, 26) the compound 2-aminoethane sulfonate or 2-aminoethane sulfonic acid also known as taurine is found in plants only in very low levels (27). The metabolic pathways for taurine and hypotaurine have not been identified in plants. The metabolic pathways that synthesize taurine and hypotaurine have only been identified in animals and bacteria. However the exogenous application of taurine has been reported to increase crop harvest, yield, and biomass, increase seedling growth and increase photosynthetic capacity of isolated plant cells and chloroplasts (28).

Unique Opportunity to Use the Promiscuous Enzyme Activity of SAD in Plants

Based on the searches of the known genome databases and published scientific reports, plants do not have a SAD gene. Plants do not have a protein with SAD activity, and they lack the metabolic intermediates of the SAD pathway. Thus there is no obvious reason that adding a SAD gene to a plant without adding the other genes in the SAD-mediated pathway will have any benefit. To date there are no reports of 3-sulfino-L-alanine in plants. If it is the case that a plant does have 3-sulfino-L-alanine it is expected that the plant could make taurine. However, in the absence of the preferred SAD substrate, 3-sulfino-L-alanine, or at low concentrations of 3-sulfino-L-alanine, SAD through promiscuous enzyme activity will use low affinity substrates and make bioactive compounds. These bioactive compounds may increase plant growth, development, and production and can be used to enhance the agronomic, nutritional, or pharmaceutical value of a plant or crop.

### SUMMARY OF THE INVENTION

The invention relates to the use of functional sulfinoalanine decarboxylase (SAD) or the promiscuous enzyme activity of SAD (collectively referred to hereinafter as SAD) in cells, particularly plant cells or algal cells. The invention also relates to methods for assembling expression and/or vector constructs and other nucleic acid molecules for transformation into plants. The invention further relates to methods to improve agronomic traits by inserting into plants a gene that encodes SAD. The transgenic plants will have increased plant growth biomass, yield, or tolerance to biotic and/or abiotic stresses. The invention also relates to transgenic plants and seed having a coding sequence for SAD stably incorporated into their genome.

In one aspect, the present invention provides an expression cassette which comprises a promoter that is operably linked to a polynucleotide that encodes SAD that is operably linked to a terminator. In one embodiment, the promoter is a promoter that is operable in a plant cell, and the terminator is a terminator operable in a plant cell. In another embodiment, the promoter is a promoter that is operable in an algal cell, and the terminator is a terminator operable in an algal cell. In a further embodiment, the promoter is a constitutive promoter or a non-constitutive promoter selected from the group consisting of a tissue-preferred promoter, a tissue-specific promoter, a cell type-specific promoter, an inducible promoter, a plant glutamate decarboxylase (GAD) promoter, a plant sulphate transporter (SULTR) promoter, or a plant glutamate receptor (GLR) promoter. In one embodiment, the polynucleotide encoding SAD has a nucleotide set forth in SEQ ID NO:1, SEQ ID NO:2, SEQ ID NO:3, SEQ ID NO:4, SEQ ID NO:5, SEQ ID NO:6, or SEQ ID NO:7. In another embodiment the polynucleotide encodes a SAD having an amino acid sequence set forth in SEQ ID NO:8, SEQ ID NO:9, SEQ ID NO:10, SEQ ID NO:11, SEQ ID NO:12, or SEQ ID NO:13. In a further embodiment, the polynucleotide encoding a SAD further comprises a subcellular location sequence that targets a subcellular location. In one embodiment, the subcellular location is an apoplast, a vacuole, a plastid, a chloroplast, a proplastid, an etioplast, a chromoplast, a mitochondrion, a peroxisome, a glyoxysome, the nucleus, a lysosome, an endomembrane system, an endoplasmic reticulum, a vesicle, or a Golgi apparatus.

In a second aspect, the present invention provides a plant or alga having stably integrated into its genome an expression cassette as described herein. In one embodiment, the invention provides a plant storage organ having stably integrated into its genome an expression cassette as described herein. In another embodiment, the plant storage unit is a seed, a tuber, a fruit or a root. In a further embodiment, the plant has increased growth, yield, or biomass, altered development, increased water-use-efficiency, increased nitrogen-use-efficiency, or increased tolerance to biotic stress (pests, pathogens, bacteria, microbes, viruses, viroids, microorganisms, invertebrates, insects, nematodes, vertebrates) or abiotic stress (osmotic stress, oxidative damage, drought, salt, cold, freezing, heat, UV light, limitations of nutrients such as nitrogen, sulfur, phosphorous or other minerals).

In a third aspect, the present invention provides a method of producing a crop of plants having stably incorporated in their genome an exogenous polynucleotide encoding SAD. In one embodiment, the method comprises growing plants from the seed described herein. In another embodiment, the method comprises multiplying plants described herein. In a further embodiment, the method comprises breeding plants described herein.

In a fourth aspect, the present invention provides a composition. In one embodiment, the composition is a pharmaceutical composition comprising an extract of a plant described herein. In another embodiment, the composition is a nutritional supplement comprising an extract of a plant described herein. In a further embodiment, the composition is an animal feed supplement comprising a component that is a plant storage organ described herein, a seed described herein or a plant described herein.

### BRIEF DESCRIPTION OF THE FIGURE

FIG. 1 shows the range of known substrates that can be used by sulfinoalanine decarboxylase (SAD). The preferred substrate for SAD is 3-sulfino-L-alanine (dark gray box). However in the absence, or at low concentrations, of 3-sulfino-L-alanine SAD through promiscuous enzyme activity (light gray box) can decarboxylate (release CO₂) other substrates such as aspartate, glutamate or cysteic acid to make beta-alanine, 4-aminobutanoate or 2-aminoethane sulfonate, respectively.

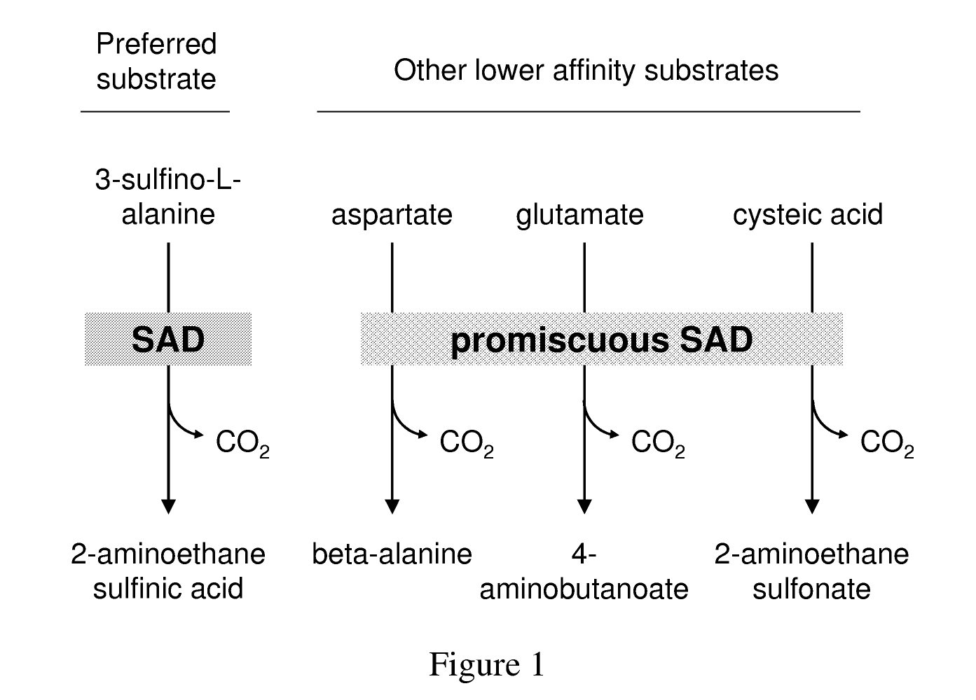

### DETAILED DESCRIPTION OF THE INVENTION

The present invention provides methods for the use of functional sulfinoalanine decarboxylase (SAD) or the promiscuous enzyme activity of SAD in cells specifically plant and algal cells. In preferred embodiments, the invention describes the genetic transformation of organisms, preferably plants, with genes that encode SAD proteins. Plants with SAD enzyme activity have improved plant growth, development and performance, that is, increased plant size, biomass, yield or tolerance to biotic or abiotic stress.

Inventive methods include the construction of plants that have advantages of improved production, such as enhanced plant growth characteristics (root mass, biomass, yield), survival characteristics and/or tolerance to environmental stresses including, but not limited to, limitations of nutrients (such as nitrogen, sulfur, phosphorous or other minerals), osmotic stress, oxidative damage, drought, salinity, cold, freezing, elevated temperature, UV light or high light intensity, and/or tolerance to biotic stresses including, but not limited to, insect or nematode feeding, bacterial or fungal infection or challenges, infection or insult from pests, pathogens, viruses, or viroids.

The invention also provides methods of using plants or plant organs with SAD genes for food supplement, feed supplement, dietary supplement, or as a component of a health supplement or therapy.

The present invention describes the methods for the synthesis of DNA constructs for the insertion of a SAD gene into plants to increase plant growth, biomass or yield. The invention describes methods for the use of polynucleotides that encode functional SAD in plants. The preferred embodiment of the invention is in plants but other organisms such as algae may be used.

Enzymes for Sulfinoalanine Decarboxylase (SAD)

Examples of amino acid sequences with SAD enzyme activity are SEQ ID NO:8, SEQ ID NO:9, SEQ ID NO:10, SEQ ID NO:11, SEQ ID NO:12, or SEQ ID NO:13. The invention is not limited to the use of these amino acid sequences. Those of ordinary skill in the art know that organisms of a wide variety of species commonly express and utilize homologous proteins, which include the insertions, substitutions and/or deletions discussed above, and effectively provide similar function. For example, the amino acid sequences of SAD from SEQ ID NO:8, SEQ ID NO:9, SEQ ID NO:10, SEQ ID NO:11, SEQ ID NO:12, or SEQ ID NO:13 may differ to a certain degree from the amino acid sequences of SAD in another species and yet have similar functionality with respect to catalytic and regulatory function Amino acid sequences comprising such variations are included within the scope of the present invention and are considered substantially or sufficiently similar to a reference amino acid sequence. Although it is not intended that the present invention be limited by any theory by which it achieves its advantageous result, it is believed that the identity between amino acid sequences that is necessary to maintain proper functionality is related to maintenance of the tertiary structure of the polypeptide such that specific interactive sequences will be properly located and will have the desired activity, and it is contemplated that a polypeptide including these interactive sequences in proper spatial context will have activity.

Another manner in which similarity may exist between two amino acid sequences is where there is conserved substitution between a given amino acid of one group, such as a non-polar amino acid, an uncharged polar amino acid, a charged polar acidic amino acid, or a charged polar basic amino acid, with an amino acid from the same amino acid group. For example, it is known that the uncharged polar amino acid serine may commonly be substituted with the uncharged polar amino acid threonine in a polypeptide without substantially altering the functionality of the polypeptide. Whether a given substitution will affect the functionality of the enzyme may be determined without undue experimentation using synthetic techniques and screening assays known to one with ordinary skill in the art.

One of ordinary skill in the art will recognize that changes in the amino acid sequences, such as individual substitutions, deletions or additions to a nucleic acid, peptide, polypeptide, or protein sequence which alters, adds or deletes a single amino acid or a small percentage of amino acids in the encoded sequence is “sufficiently similar” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, SAD activity is generally at least 40%, 50%, 60%, 70%, 80% or 90%, preferably 60-90% of the native protein for the native substrate. Tables of conserved substitution provide lists of functionally similar amino acids.

The following three groups each contain amino acids that are conserved substitutions for one another: (1) Alanine (A), Serine (S), Threonine (T); (2) Aspartic acid (D), Glutamic acid (E); and (3) Asparagine (N), Glutamine (Q).

Suitable Polynucleotides for SAD

As examples, suitable polynucleotides encoding SAD proteins are described below. The invention is not limited to use of these sequences. However, suitable polynucleotides for SAD are provided in SEQ ID NO:1, SEQ ID NO:2, SEQ ID NO:3, SEQ ID NO:4, SEQ ID NO:5, SEQ ID NO:6 and SEQ ID NO:7. Other suitable polynucleotides for use in accordance with the invention may be obtained by the identification of polynucleotides that selectively hybridize to the polynucleotides of SEQ ID NO:1, SEQ ID NO:2, SEQ ID NO:3, SEQ ID NO:4, SEQ ID NO:5, SEQ ID NO:6 or SEQ ID NO:7 by hybridization under low stringency conditions, moderate stringency conditions, or high stringency conditions. Still other suitable polynucleotides for use in accordance with the invention may be obtained by the identification of polynucleotides that have substantial identity of the nucleic acid of SEQ ID NO:1, SEQ ID NO:2, SEQ ID NO:3, SEQ ID NO:4, SEQ ID NO:5, SEQ ID NO:6 and SEQ ID NO:7 when it used as a reference for sequence comparison or polynucleotides that encode polypeptides that have substantial identity to amino acid sequence of SEQ ID NO:8, SEQ ID NO:9, SEQ ID NO:10, SEQ ID NO:11, SEQ ID NO:12, or SEQ ID NO:13 when it is used as a reference for sequence comparison.

Another embodiment of the invention is a polynucleotide (e.g., a DNA construct) that encodes a protein that functions as SAD and selectively hybridizes to either SEQ ID NO:1, SEQ ID NO:2, SEQ ID NO:3, SEQ ID NO:4, SEQ ID NO:5, SEQ ID NO:6 or SEQ ID NO:7. Selectively hybridizing sequences typically have at least 40% sequence identity, preferably 60-90% sequence identity, and most preferably 100% sequence identity with each other.

Database searches and homology searches of genome and nucleotide databases identify similar DNA or RNA molecules based on the alignment of nucleotides using algorithms or computer programs and these techniques well known to those of skill in the art. In accordance with the invention other suitable polynucleotides for use may be obtained by the in silico identification of polynucleotides for SAD with at least 50% sequence identity, preferably 60-80% sequence identity, and most preferably 81% to 100% sequence identity with SEQ ID NO:1, SEQ ID NO:2, SEQ ID NO:3, SEQ ID NO:4, SEQ ID NO:5, SEQ ID NO:6 or SEQ ID NO:7.

Another embodiment of the invention is a polynucleotide that encodes a polypeptide that has substantial identity to the amino acid sequence of SEQ ID NO:8, SEQ ID NO:9, SEQ ID NO:10, SEQ ID NO:11, SEQ ID NO:12, or SEQ ID NO:13 Substantial identity of amino acid sequences for these purposes normally means sequence identity of between 50-100%, preferably at least 55%, preferably at least 60%, more preferably at least 70%, 80%, 90%, and most preferably at least 95%.

Database and homology searches can also be used to alignment polypeptides. In accordance with the invention other suitable polypeptides for use may be obtained by the in silico identification of polypeptides for SAD with at least 50% sequence identity, preferably 60-80% sequence identity, and most preferably 81% to 100% sequence identity with of SEQ ID NO:8, SEQ ID NO:9, SEQ ID NO:10, SEQ ID NO:11, SEQ ID NO:12, or SEQ ID NO:13.

The process of encoding a specific amino acid sequence may involve DNA sequences having one or more base changes (i.e., insertions, deletions, substitutions) that do not cause a change in the encoded amino acid, or which involve base changes which may alter one or more amino acids, but do not eliminate the functional properties of the polypeptide encoded by the DNA sequence.

It is therefore understood that the invention encompasses more than the specific polynucleotides encoding the proteins described herein. For example, modifications to a sequence, such as deletions, insertions, or substitutions in the sequence, which produce “silent” changes that do not substantially affect the functional properties of the resulting polypeptide are expressly contemplated by the present invention. Furthermore, because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill in the art will recognize that each amino acid has more than one codon, except for methionine and tryptophan that ordinarily have the codons AUG and UGG, respectively. It is known by those of ordinary skill in the art, “universal” code is not completely universal. Some mitochondrial and bacterial genomes diverge from the universal code, e.g., some termination codons in the universal code specify amino acids in the mitochondria or bacterial codes. Thus each silent variation of a nucleic acid, which encodes a polypeptide of the present invention, is implicit in each described polypeptide sequence and incorporated in the descriptions of the invention.

It is understood that alterations in a nucleotide sequence, which reflect the degeneracy of the genetic code, or which result in the production of a chemically equivalent amino acid at a given site, are contemplated. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a biologically equivalent product.

Nucleotide changes which result in alteration of the amino-terminal and carboxy-terminal portions of the encoded polypeptide molecule would also not generally be expected to alter the activity of the polypeptide. In some cases, it may in fact be desirable to make mutations in the sequence in order to study the effect of alteration on the biological activity of the polypeptide. Each of the proposed modifications is well within the routine skill in the art.

When the nucleic acid is prepared or altered synthetically, one of ordinary skill in the art can take into account the known codon preferences for the intended host where the nucleic acid is to be expressed. For example, although nucleic acid sequences of the present invention may be expressed in both monocotyledonous and dicotyledonous plant species, sequences can be modified to account for the specific codon preferences and GC-content preferences of monocotyledonous plants or dicotyledonous plants, as these preferences have been shown to differ (29). In addition, modifications in the nucleotide sequence around the start codon can optimize expression in transgenic plants (30, 31). See, for example, U.S. Pat. Nos. 5,380,831, 5,436,391, and 7,205,453 and U.S. Patent Application Publication Nos. 2006/0218670 and 2006/0248616. An alternative approach to the generation of variants of the sequences is to use random recombination techniques such as “DNA shuffling” as disclosed in U.S. Pat. Nos. 5,605,793; 5,811,238; 5,830,721 and 5,837,458; and International Applications WO 98/31837 and WO 99/65927. An alternative method of to modify the sequences is by rapid molecular evolution methods such as a staggered extension process as disclosed in U.S. Pat. No. 5,965,408 and International Application WO 98/42832.

Cloning Techniques

For purposes of promoting an understanding of the principles of the invention, reference will now be made to particular embodiments of the invention and specific language will be used to describe the same. The materials, methods and examples are illustrative only and not limiting. Unless mentioned otherwise, the techniques employed or contemplated herein are standard methodologies well known to one of ordinary skill in the art. Specific terms, while employed below and defined at the end of this section, are used in a descriptive sense only and not for purposes of limitation. The practice of the present invention will employ, unless otherwise indicated, conventional techniques of botany, microbiology, tissue culture, molecular biology, chemistry, biochemistry and recombinant DNA technology, which are within the skill of the art (32-39).

A suitable polynucleotide for use in accordance with the invention may be obtained by cloning techniques using cDNA or genomic libraries, DNA, or cDNA from bacteria which are available commercially or which may be constructed using standard methods known to persons of ordinary skill in the art. Suitable nucleotide sequences may be isolated from DNA libraries obtained from a wide variety of species by means of nucleic acid hybridization or amplification methods, such as polymerase chain reaction (PCR) procedures, using as probes or primers nucleotide sequences selected in accordance with the invention.

Furthermore, nucleic acid sequences may be constructed or amplified using chemical synthesis. The product of amplification is termed an amplicon. Moreover, if the particular nucleic acid sequence is of a length that makes chemical synthesis of the entire length impractical, the sequence may be broken up into smaller segments that may be synthesized and ligated together to form the entire desired sequence by methods known in the art. Alternatively, individual components or DNA fragments may be amplified by PCR and adjacent fragments can be amplified together using fusion-PCR (40), overlap-PCR (41) or chemical (de novo) synthesis (42-46) by methods known in the art. Gene constructs can be routinely synthesized commercially by several vendors that include but are not limited to Bio Basic, Bio-Synthesis, DNA2.0, Epoch Life Science, GENEART, GENEWIZ, GenScript, Hyglos GmbH, Integrated DNA Technologies, Invitrogen, or Primm Biotech.

A suitable polynucleotide for use in accordance with the invention may be constructed by recombinant DNA technology, for example, by cutting or splicing nucleic acids using restriction enzymes and mixing with a cleaved (cut with a restriction enzyme) vector with the cleaved insert (DNA of the invention) and ligating using DNA ligase. Alternatively amplification techniques, such as PCR, can be used, where restriction sites are incorporated in the primers that otherwise match the nucleotide sequences (especially at the 3′ ends) selected in accordance with the invention. The desired amplified recombinant molecule is cut or spliced using restriction enzymes and mixed with a cleaved vector and ligated using DNA ligase. In another method, after amplification of the desired recombinant molecule, DNA linker sequences are ligated to the 5′ and 3′ ends of the desired nucleotide insert with ligase, the DNA insert is cleaved with a restriction enzyme that specifically recognizes sequences present in the linker sequences and the desired vector. The cleaved vector is mixed with the cleaved insert, and the two fragments are ligated using DNA ligase. In yet another method, the desired recombinant molecule is amplified with primers that have recombination sites (e.g. Gateway) incorporated in the primers that otherwise match the nucleotide sequences selected in accordance with the invention. The desired amplified recombinant molecule is mixed with a vector containing the recombination site and recombinase. The two molecules are bond together by recombination.

The recombinant expression cassette or DNA construct includes a promoter that directs transcription in a plant cell, operably linked to the polynucleotide encoding SAD. In various aspects of the invention described herein, a variety of different types of promoters are described and used. As used herein, a polynucleotide is “operably linked” to a promoter or other nucleotide sequence when it is placed into a functional relationship with a promoter or other nucleotide sequence. The functional relationship between a promoter and a desired polynucleotide insert typically involves the polynucleotide and the promoter sequences being contiguous such that transcription of the polynucleotide sequence will be facilitated. Two nucleic acid sequences are further said to be operably linked if the nature of the linkage between the two sequences does not (1) result in the introduction of a frame-shift mutation; (2) interfere with the ability of the promoter region sequence to direct the transcription of the desired nucleotide sequence, or (3) interfere with the ability of the desired nucleotide sequence to be transcribed by the promoter sequence region. Typically, the promoter element is upstream (i.e., at the 5′ end) of the nucleic acid insert coding sequence.

While a promoter sequence can be ligated to a coding sequence prior to insertion into a vector, in other embodiments, a vector is selected that includes a promoter operable in the host cell into which the vector is to be inserted. In addition, certain preferred vectors have a region that codes a ribosome binding site positioned between the promoter and the site at which the DNA sequence is inserted so as to be operatively associated with the DNA sequence of the invention to produce the desired polypeptide, i.e., the DNA sequence of the invention in-frame.

Suitable Promoters

Known to those of ordinary skill in the art are a wide variety of promoters and other regulatory elements that can be used alone or in combination with promoters. A wide variety of promoters that direct transcription in plants cells can be used in connection with the present invention. For purposes of describing the present invention, promoters are divided into two types, namely, constitutive promoters and non-constitutive promoters. Constitutive promoters are classified as providing a range of constitutive expression; some are weak constitutive promoters and others are strong constitutive promoters. Non-constitutive promoters include tissue-preferred promoters, tissue-specific promoters, cell-type specific promoters, and inducible-promoters.

Of particular interest in certain embodiments of the present invention are inducible-promoters that respond to various forms of environmental stresses, or other stimuli, including, for example, mechanical shock, heat, cold, salt, flooding, drought, salt, anoxia, pathogens, such as bacteria, fungi, and viruses, and nutritional deprivation, including deprivation during times of flowering and/or fruiting, and other forms of plant stress. For example, the promoter selected in alternate forms of the invention can be a promoter induced by one or more factors, including but not limited to, abiotic stresses such as wounding, cold, dessication, ultraviolet-B (47), heat shock (48) or other heat stress, drought stress or water stress. The promoter may further be a promoter induced by biotic stresses including, but not limited to, pathogen stress, such as stress induced by a virus (49) or fungi (50, 51), stresses induced as part of the plant defense pathway (52) or by other environmental signals, such as light (53), carbon dioxide (54, 55), hormones or other signaling molecules such as auxin, hydrogen peroxide and salicylic acid (56, 57), sugars and gibberellin (58) or abscissic acid and ethylene (59).

In other embodiments of the invention, tissue-specific promoters are used. Tissue-specific expression patterns are controlled by tissue- or stage-specific promoters that include, but are not limited to, fiber-specific, green tissue-specific, root-specific, stem-specific, and flower-specific. Examples of the utilization of tissue-specific expression include, but are not limited to, the expression in leaves of the desired peptide for the protection of plants against foliar pathogens, the expression in roots of the desired peptide for the protection of plants against root pathogens, and the expression in roots or seedlings of the desired peptide for the protection of seedlings against soil-borne pathogens. In many cases, however, protection against more than one type of pathogen may be sought, and expression in multiple tissues will be desirable.

There are suitable promoters for root-specific expression (60, 61). A selected promoter can be an endogenous promoter, i.e. a promoter native to the species or cell type being transformed. Alternatively, the promoter can be a foreign promoter, which promotes transcription of a length of DNA of viral, microbial, bacterial or eukaryotic origin, invertebrates, vertebrates including those from plants or plant viruses. For example, in certain preferred embodiments, the promoter may be of viral origin, including a cauliflower mosaic virus promoter (CaMV), such as CaMV 35S or 19S, a figwort mosaic virus promoter (FMV 35S), or the coat protein promoter of tobacco mosaic virus (TMV). The promoter may further be a promoter for the small subunit of ribulose-1,3-biphosphate carboxylase. Promoters of bacterial origin (microbe promoters) include the octopine synthase promoter, the nopaline synthase promoter and other promoters derived from native Ti plasmids (62).

The promoters may further be selected such that they require activation by other elements known to those of ordinary skill in the art, so that production of the protein encoded by the nucleic acid sequence insert may be regulated as desired. In one embodiment of the invention, a DNA construct comprising a non-constitutive promoter operably linked to a polynucleotide encoding the desired polypeptide of the invention is used to make a transformed plant that selectively increases the level of the desired polypeptide of the invention in response to a signal. The term “signal” is used to refer to a condition, stress or stimulus that results in or causes a non-constitutive promoter to direct expression of a coding sequence operably linked to it. To make such a plant in accordance with the invention, a DNA construct is provided that includes a non-constitutive promoter operably linked to a polynucleotide encoding the desired polypeptide of the invention. The construct is incorporated into a plant genome to provide a transformed plant that expresses the polynucleotide in response to a signal.

In alternate embodiments of the invention, the selected promoter is a tissue-preferred promoter, a tissue-specific promoter, a cell-type-specific promoter, an inducible promoter or other type of non-constitutive promoter. It is readily apparent that such a DNA construct causes a plant transformed thereby to selectively express the gene for the desired polypeptide of the invention. Therefore under specific conditions or in certain tissue- or cell-types the desired polypeptide will be expressed. The result of this expression in the plant depends upon the activity of the promoter and in some cases the conditions of the cell or cells in which it is expressed.

It is understood that the non-constitutive promoter does not continuously produce the transcript or RNA of the invention. But in this embodiment the selected promoter for inclusion of the invention advantageously induces or increases transcription of gene for the desired polypeptide of the invention in response to a signal, such as an environmental cue or other stress signal including biotic and/or abiotic stresses or other conditions.

In another embodiment of the invention, a DNA construct comprising a plant GAD promoter operably linked to polynucleotides that encode the desired polypeptide of the invention is used to make a transformed plant that selectively increases the transcript or RNA of the desired polypeptide of the invention in the same cells, tissues, and under the environmental conditions that express a plant glutamate decarboxylase. It is understood to those of ordinary skill in the art that the regulatory sequences that comprise a plant promoter driven by RNA polymerase II reside in the region approximately 2900 to 1200 basepairs (bp) up-stream (5′) of the translation initiation site or start codon (ATG). For example, the full-length promoter for the nodule-enhanced PEP carboxylase from alfalfa is 1277 bps prior to the start codon (63), the full-length promoter for cytokinin oxidase from orchid is 2189 bps prior to the start codon (64), the full-length promoter for ACC oxidase from peach is 2919 bps prior to the start codon (65), full-length promoter for cytokinin oxidase from orchid is 2189 bps prior to the start codon, full-length promoter for glutathione peroxidase1 from Citrus sinensis is 1600 bps prior to the start codon (66), and the full-length promoter for glucuronosyltransferase from cotton is 1647 bps prior to the start codon (67). Most full-length promoters are 1700 bps prior to the start codon. The accepted convention is to describe this region (promoter) as −1700 to −1 bp, where the numbers designate the number of bps prior to the “A” in the start codon. In this embodiment of the invention the region of −2000 to −1 bps 5′ to a plant GAD is operably linked to a polynucleotide for the said encoded peptide to make a transformed plant that selectively expresses the polynucleotide or increases the level of the said protein where the plant GAD is expressed or accumulates. Suitable plant GAD promoters include but are not limited to the −2000 to −1 bp region of the GAD genes in Arabidopsis thaliana (AtGAD) (68), petunia (69), tomato (70), tobacco (71), rice (72), barely, poplar, soybean, mustard, orange, Medicago truncatula, grape and pine. Those of ordinary skill in the art can either digest the desired region using restriction enzymes and ligase to clone the plant GAD promoters or use amplification techniques, such as PCR, with the incorporation of restriction or recombination sites to clone the plant GAD promoters 5′ to the desired polynucleotide. For the purpose of this invention, a plant GAD promoter is the region upstream (5′) to the start codon between −200 to −1 bps, preferably at least between −500 to −1 bps, preferably at least between −1000 to −1 bps, more preferably at least between −1500 to −1 bps, and most preferably at −2000 to −1 bps.

In another embodiment of the invention, a DNA construct comprising a plant glutamate receptor promoter operably linked to polynucleotides that encode the desired polypeptide of the invention is used to make a transformed plant that selectively increases the transcript or RNA of the desired polypeptide of the invention in the same cells, tissues, and under the environmental conditions that express a plant glutamate receptor. It is understood to those of ordinary skill in the art that the regulatory sequences that comprise a plant promoter driven by RNA polymerase II reside in the region approximately 2900 to 1200 bps up-stream (5′) of the translation initiation site or start codon (ATG). Suitable plant glutamate receptor promoters include but are not limited to the −2000 to −1 bp region of the plant glutamate receptor genes in Arabidopsis thaliana (AtGLRs or AtGluRs) and rice. The promoters for the following AtGLRs genes, 1.1, 2.1, 3.1 (73), 3.2 (note this is designated as GLR2 in the manuscript) (74), and 3.4 (75) have been shown to control specific cell-type, tissue-type, developmental and environmental expression patterns in plants. Those of ordinary skill in the art can either digest the desired region using restriction enzymes and ligase to clone the plant glutamate promoters or use amplification techniques, such as PCR, with the incorporation of restriction or recombination sites to clone the plant glutamate receptor promoters 5′ to the desired polynucleotide. For the purpose of this invention, a plant glutamate receptor promoter is the region upstream (5′) to the start codon between −200 to −1 bps, preferably at least between −500 to −1 bps, preferably at least between −1000 to −1 bps, more preferably at least between −1500 to −1 bps, and most preferably at −2000 to −1 bps.

In another embodiment of the invention, a DNA construct comprising a plant sulphate transporter promoter operably linked to polynucleotides that encode the desired polypeptide of the invention is used to make a transformed plant that selectively increases the transcript or RNA of the desired polypeptide of the invention in the same cells, tissues, and under the environmental conditions that express a plant sulphate transporter. It is understood to those of ordinary skill in the art that the regulatory sequences that comprise a plant promoter driven by RNA polymerase II reside in the region approximately 2900 to 1200 bps up-stream (5′) of the translation initiation site or start codon (ATG). Suitable plant sulphate transporter promoters include but are not limited to the −2000 to −1 bp region of the plant sulphate transporter genes in Arabidopsis thaliana (SULTR or AtSULTR). The promoters for the following SULTR genes, SULTR1;1, SULTR1;2 (76), SULTR 1;3; (77), SULTR2;1 (78), and SULTR3;5 (79) have been shown to control specific cell-type, tissue-type, developmental and environmental expression patterns in plants. Those of ordinary skill in the art can either digest the desired region using restriction enzymes and ligase to clone the plant glutamate promoters or use amplification techniques, such as PCR, with the incorporation of restriction or recombination sites to clone the plant sulphate transporter promoters 5′ to the desired polynucleotide. For the purpose of this invention, a plant sulphate transporter promoter is the region upstream (5′) to the start codon between −200 to −1 bps, preferably at least between −500 to −1 bps, preferably at least between −1000 to −1 bps, more preferably at least between −1500 to −1 bps, and most preferably at −2000 to −1 bps.

Suitable Vectors

A wide variety of vectors may be employed to transform a plant, plant cell or other cells with a construct made or selected in accordance with the invention, including high- or low-copy number plasmids, phage vectors and cosmids. Such vectors, as well as other vectors, are well known in the art. Representative T-DNA vector systems (62, 80) and numerous expression cassettes and vectors and in vitro culture methods for plant cell or tissue transformation and regeneration of plants are known and available (81). The vectors can be chosen such that operably linked promoter and polynucleotides that encode the desired polypeptide of the invention are incorporated into the genome of the plant. Although the preferred embodiment of the invention is expression in plants or plant cells, other embodiments may include expression in prokaryotic or eukaryotic photosynthetic organisms, microbes, invertebrates or vertebrates.

It is known by those of ordinary skill in the art that there exist numerous expression systems available for expression of a nucleic acid encoding a protein of the present invention. There are many commercially available recombinant vectors to transform a host plant or plant cell. Standard molecular and cloning techniques (36, 39, 82) are available to make a recombinant expression cassette that expresses the polynucleotide that encodes the desired polypeptide of the invention. No attempt to describe in detail the various methods known for the expression of proteins in prokaryotes or eukaryotes will be made. In brief, the expression of isolated nucleic acids encoding a protein of the present invention will typically be achieved by operably linking, for example, the DNA or cDNA to a promoter, followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high-level expression of a cloned gene, it is desirable to construct expression vectors that contain, at the minimum, a strong promoter, such as ubiquitin, to direct transcription, a ribosome-binding site for translational initiation, and a transcription/translation terminator.

One of ordinary skill in the art recognizes that modifications could be made to a protein of the present invention without diminishing its biological activity. Some modifications may be made to facilitate the cloning, expression, targeting or to direct the location of the polypeptide in the host, or for the purification or detection of the polypeptide by the addition of a “tag” as a fusion protein. Such modifications are well known to those of skill in the art and include, for example, a methionine added at the amino terminus to provide an initiation site, additional amino acids (tags) placed on either terminus to create a tag, additional nucleic acids to insert a restriction site or a termination.

In addition to the selection of a suitable promoter, the DNA constructs require an appropriate transcriptional terminator to be attached downstream of the desired gene of the invention for proper expression in plants. Several such terminators are available and known to persons of ordinary skill in the art. These include, but are not limited to, the tml from CaMV and E9 from rbcS. Another example of a terminator sequence is the polyadenlyation sequence from the bovine growth hormone gene. A wide variety of available terminators known to function in plants can be used in the context of this invention. Vectors may also have other control sequence features that increase their suitability. These include an origin of replication, enhancer sequences, ribosome binding sites, RNA splice sites, polyadenylation sites, selectable markers and RNA stability signal. Origin of replication is a gene sequence that controls replication of the vector in the host cell. Enhancer sequences cooperate with the promoter to increase expression of the polynucleotide insert coding sequence. Enhancers can stimulate promoter activity in host cell. An example of specific polyadenylation sequence in higher eukaryotes is ATTTA. Examples of plant polyadenylation signal sequences are AATAAA or AATAAT. RNA splice sites are sequences that ensure accurate splicing of the transcript. Selectable markers usually confer resistance to an antibiotic, herbicide or chemical or provide color change, which aid the identification of transformed organisms. The vectors also include a RNA stability signal, which is a 3′-regulatory sequence element that increases the stability of the transcribed RNA (83, 84).

In addition, polynucleotides that encode a SAD can be placed in the appropriate plant expression vector used to transform plant cells. The polypeptide can then be isolated from plant callus, or the transformed cells can be used to regenerate transgenic plants. Such transgenic plants can be harvested, and the appropriate tissues can be subjected to large-scale protein extraction and purification techniques.

The vectors may include another polynucleotide insert that encodes a peptide or polypeptide used as a “tag” to aid in purification or detection of the desired protein. The additional polynucleotide is positioned in the vector such that upon cloning and expression of the desired polynucleotide a fusion, or chimeric, protein is obtained. The tag may be incorporated at the amino or carboxy terminus. If the vector does not contain a tag, persons with ordinary skill in the art know that the extra nucleotides necessary to encode a tag can be added with the ligation of linkers, adaptors, or spacers or by PCR using designed primers. After expression of the peptide the tag can be used for purification using affinity chromatography, and if desired, the tag can be cleaved with an appropriate enzyme. The tag can also be maintained, not cleaved, and used to detect the accumulation of the desired polypeptide in the protein extracts from the host using western blot analysis. In another embodiment, a vector includes the polynucleotide for the tag that is fused in-frame to the polynucleotide that encodes a functional SAD to form a fusion protein. The tags that may be used include, but are not limited to, Arg-tag, calmodulin-binding peptide, cellulose-binding domain, DsbA, c-myc-tag, glutathione S-transferase, FLAG-tag, HAT-tag, His-tag, maltose-binding protein, NusA, S-tag, SBP-tag, Strep-tag, and thioredoxin (Trx-Tag). These are available from a variety of manufacturers Clontech Laboratories, Takara Bio Company GE Healthcare, Invitrogen, Novagen Promega and QIAGEN.

The vector may include another polynucleotide that encodes a signal polypeptide or signal sequence to direct the desired polypeptide in the host cell, so that the polypeptide accumulates in a specific cellular compartment, subcellular compartment, or membrane. The specific cellular compartments include the apoplast, vacuole, plastids chloroplast, mitochondrion, peroxisomes, secretory pathway, lysosome, endoplasmic reticulum, nucleus or Golgi apparatus. A signal polypeptide or signal sequence is usually at the amino terminus and normally absent from the mature protein due to protease that removes the signal peptide when the polypeptide reaches its final destination. Signal sequences can be a primary sequence located at the N-terminus (85-88), C-terminus (89, 90) or internal (91, 92) or tertiary structure (93). If a signal polypeptide or signal sequence to direct the polypeptide does not exist on the vector, it is expected that those of ordinary skill in the art can incorporate the extra nucleotides necessary to encode a signal polypeptide or signal sequence by the ligation of the appropriate nucleotides or by PCR. Those of ordinary skill in the art can identify the nucleotide sequence of a signal polypeptide or signal sequence using computational tools. There are numerous computational tools available for the identification of targeting sequences or signal sequence. These include, but are not limited to, TargetP (94, 95), iPSORT (96), SignalP (97), PrediSi (98), ELSpred (99) HSLpred (100) and PSLpred (101), MultiLoc (102), SherLoc (103), ChloroP (104), MITOPROT (105), Predotar (106) and 3D-PSSM (107). Additional methods and protocols are discussed in the literature (102).

Transformation of Host Cells

Transformation of a plant can be accomplished in a wide variety of ways within the scope of a person of ordinary skill in the art. In one embodiment, a DNA construct is incorporated into a plant by (i) transforming a cell, tissue or organ from a host plant with the DNA construct; (ii) selecting a transformed cell, cell callus, somatic embryo, or seed which contains the DNA construct; (iii) regenerating a whole plant from the selected transformed cell, cell callus, somatic embryo, or seed; and (iv) selecting a regenerated whole plant that expresses the polynucleotide. Many methods of transforming a plant, plant tissue or plant cell for the construction of a transformed cell are suitable. Once transformed, these cells can be used to regenerate transgenic plants (108).

Those of ordinary skill in the art can use different plant gene transfer techniques including, but not limited to, electroporation (109-113), microinjection (114, 115), lipofection (116), liposome or spheroplast fusions (117-119), Agrobacterium (120), direct gene transfer (121), T-DNA mediated transformation of monocots (122), T-DNA mediated transformation of dicots) (123, 124), microprojectile bombardment or ballistic particle acceleration (125-128), chemical transfection including CaCl₂ precipitation, polyvinyl alcohol, or poly-L-ornithine (129), silicon carbide whisker methods (130, 131), laser methods (132, 133), sonication methods (134-136), polyethylene glycol methods (137), vacuum infiltration (138), and transbacter (139).

In one embodiment of the invention, a transformed host cell may be cultured to produce a transformed plant. In this regard, a transformed plant can be made, for example, by transforming a cell, tissue or organ from a host plant with an inventive DNA construct; selecting a transformed cell, cell callus, somatic embryo, or seed which contains the DNA construct; regenerating a whole plant from the selected transformed cell, cell callus, somatic embryo, or seed; and selecting a regenerated whole plant that expresses the polynucleotide.

A wide variety of host cells may be used in the invention, including prokaryotic and eukaryotic host cells. These cells or organisms may include microbes, invertebrate, vertebrates or photosynthetic organisms. Preferred host cells are eukaryotic, preferably plant cells, such as those derived from monocotyledons, such as corn, rice, sugarcane, wheat, sorghum, bent grass, rye grass, Bermuda grass, Blue grass, Fescue, and duckweed, or dicotyledons, including canola, cotton, camelina, lettuce, rapeseed, radishes, cabbage, sugarbeet, peppers, broccoli, potatoes and tomatoes, and legumes such as soybeans and bush beans.

The foregoing methods for transformation are typically used for producing a transgenic variety in which the expression cassette is stably incorporated. After the expression cassette is stably incorporated in transgenic plants, it can be transferred to other plants by sexual crossing. In one embodiment, the transgenic variety could then be crossed with another (non-transformed or transformed) variety in order to produce a new transgenic variety. Alternatively, a genetic trait which has been engineered into a particular cotton line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties which do not contain that gene. As used herein, “crossing” can refer to a simple X by Y cross, or the process of backcrossing, depending on the context. Any of a number of standard breeding techniques can be used, depending upon the species to be crossed.

Once transgenic plants are produced, the plants themselves can be cultivated in accordance with conventional procedures. Transgenic seeds can be recovered from the transgenic plants. These seeds can then be planted in the soil and cultivated using conventional procedures to produce transgenic plants. The cultivated transgenic plants will express the DNA of interest in a tissue-preferred or tissue-specific manner as described herein.

One embodiment of the invention is a method to produce a functional sulfinoalanine decarboxylase (SAD) by the following steps:
a. operably link a promoter to the 5′ end of the polynucleotide for a SAD gene;
b. operably link a terminator to the 3′ end of the promoter-gene assembled in step 1 above;
c. insert the promoter-gene-terminator assembled in steps 1 and 2 above into a vector; and
d. transform the vector containing the construct into a plant or algal cell.

Suitable Plants

The methods described above may be applied to transform a wide variety of plants, including decorative or recreational plants or crops, but are particularly useful for treating commercial and ornamental crops. Examples of plants that may be transformed in the present invention include, but are not limited to, Acacia, alfalfa, algae, aneth, apple, apricot, artichoke, arugula, asparagus, avocado, banana, barley, beans, beech, beet, Bermuda grass, bent grass, blackberry, blueberry, Blue grass, broccoli, Brussels sprouts, cabbage, camelina, canola, cantaloupe, carrot, cassava, cauliflower, celery, cherry, chicory, cilantro, citrus, clementines, coffee, corn, cotton, cucumber, duckweed, Douglas fir, eggplant, endive, escarole, eucalyptus, fennel, fescue, figs, forest trees, garlic, gourd, grape, grapefruit, honey dew, jatropha, jicama, kiwifruit, lettuce, leeks, lemon, lime, Loblolly pine, maize, mango, melon, mushroom, nectarine, nut, oat, okra, onion, orange, an ornamental plant, palm, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, rye grass, seaweed, scallion, sorghum, Southern pine, soybean, spinach, squash, strawberry, sugarbeet, sugarcane, sunflower, sweet potato, sweetgum, switchgrass, tangerine, tea, tobacco, tomato, turf, turnip, a vine, watermelon, wheat, yams, and zucchini. Other suitable hosts include bacteria, fungi, algae and other photosynthetic organisms, and animals including vertebrate and invertebrates.

Once transformed, the plant may be treated with other “active agents” either prior to or during the exposure of the plant to stress to further decrease the effects of plant stress. “Active agent,” as used herein, refers to an agent that has a beneficial effect on the plant or increases production of amino acid production by the plant. For example, the agent may have a beneficial effect on the plant with respect to nutrition, and the resistance against, or reduction of, the effects of plant stress. Some of these agents may be precursors of end products for reaction catalyzed by SAD. These compounds could promote growth, development, biomass and yield, and change in metabolism. In addition to the twenty amino acids that are involved in protein synthesis specifically sulfur containing amino acids methionine, and cysteine, other amino acids such as glutamate, glutamine, serine, alanine and glycine, sulfur containing compounds such as fertilizer, sulfite, sulfide, sulfate, taurine, hypotaurine, cysteate, 2-sulfacetaldehyde, homotaurine, homocysteine, cystathionine, N-acetyl thiazolidine 4 carboxylic acid (ATCA), glutathione, or bile, or other non-protein amino acids, such as GABA, citrulline and ornithine, or other nitrogen containing compounds such as polyamines may also be used to activate SAD or promote SAD activity. Depending on the type of gene construct or recombinant expression cassette, other metabolites and nutrients may be used to activate SAD activity. These include, but are not limited to, sugars, carbohydrates, lipids, oligopeptides, mono-(glucose, arabinose, fructose, xylose, and ribose) di-(sucrose and trehalose) and polysaccharides, carboxylic acids (succinate, malate and fumarate) and nutrients such as phosphate, molybdate, or iron.

Accordingly, the active agent may include a wide variety of fertilizers, pesticides and herbicides known to those of ordinary skill in the art (140). Other greening agents fall within the definition of “active agent” as well, including minerals such as calcium, magnesium and iron. The pesticides protect the plant from pests or disease and may be either chemical or biological and include fungicides, bactericides, insecticides and anti-viral agents as known to those of ordinary skill in the art.

In some embodiments properties of a transgenic plant are altered using an agent which increases sulfur concentration in cells of the transgenic plant, such as fertilizer, sulfur, sulfite, sulfide, sulfate, taurine, hypotaurine, homotaurine, cysteate, 2-sulfacetaldehyde, N-acetyl thiazolidine 4 carboxylic acid (ATCA), glutathione, and bile. In other embodiments, the agent increases nitrogen concentration. Amino acids either naturally occurring in proteins (e.g., cysteine, methionine, glutamate, glutamine, serine, alanine, or glycine) or which do no naturally occur in proteins (e.g., GABA, citrulline, or ornithine) and/or polyamines can be used for this purpose.

Expression in Prokaryotes

The use of prokaryotes as hosts includes strains of E. coli. However, other microbial strains including, but not limited to, Bacillus (141) and Salmonella may also be used. Commonly used prokaryotic control sequences include promoters for transcription initiation, optionally with an operator, along with ribosome binding site sequences. Commonly used prokaryotic promoters include the beta lactamase (142), lactose (142), and tryptophan (143) promoters. The vectors usually contain selectable markers to identify transfected or transformed cells. Some commonly used selectable markers include the genes for resistance to ampicillin, tetracycline, or chloramphenicol. The vectors are typically a plasmid or phage. Bacterial cells are transfected or transformed with the plasmid vector DNA. Phage DNA can be infected with phage vector particles or transfected with naked phage DNA. The plasmid and phage DNA for the vectors are commercially available from numerous vendors known to those of ordinary skill in the art.

Pharmaceutical Compositions

Extracts from transgenic plants expressing SAD may be used as pharmaceutical compositions. Pharmaceutically acceptable vehicles of the extracts are tablets, capsules, gel, ointment, film, patch, powder or dissolved in liquid form.

Nutritional Supplements and Feeds

Transgenic plants containing SAD may be consumed or used to make extracts for nutritional supplements. Transgenic plant parts that express SAD may be used for human consumption. The plant parts may include but are not limited to leaves, stalks, stems, tubers, stolons, roots, petioles, cotyledons, seeds, fruits, grain, strover, nuts, flowers, petioles, pollen, buds, or pods. Extracts from transgenic plants containing SAD may be used as nutritional supplements, as an antioxidant or to improve physical or mental performance. The extracts may be used in the form of a liquid, powder, capsule or tablet.

Transgenic plants containing SAD may be used as fish or animal feed or used to make extracts for the supplementation of animal feed. Plant parts that have SAD may be used as animal or fish feed include but are not limited to leaves, stalks, stems, tubers, stolons, roots, petioles, cotyledons, seeds, fruits, grain, strover, nuts, flowers, petioles, buds, pods, or husks. Extracts from transgenic plants expressing SAD may be used as feed supplements in the form of a liquid, powder, capsule or tablet.

### DEFINITIONS

Unless defined otherwise, all technical and scientific terms used herein have the same meaning as is commonly understood by one of skill in the art to which the invention belongs.

The term “polynucleotide” refers to a natural or synthetic linear and sequential array of nucleotides and/or nucleosides, including deoxyribonucleic acid, ribonucleic acid, and derivatives thereof. It includes chromosomal DNA, self-replicating plasmids, infectious polymers of DNA or RNA and DNA or RNA that performs a primarily structural role. Unless otherwise indicated, nucleic acids or polynucleotide are written left to right in 5′ to 3′ orientation, Nucleotides are referred to by their commonly accepted single-letter codes. Numeric ranges are inclusive of the numbers defining the range.

The terms “amplified” and “amplification” refer to the construction of multiple copies of a nucleic acid sequence or multiple copies complementary to the nucleic acid sequence using at least one of the nucleic acid sequences as a template. Amplification can be achieved by chemical synthesis using any of the following methods, such as solid-phase phosphoramidate technology or the polymerase chain reaction (PCR). Other amplification systems include the ligase chain reaction system, nucleic acid sequence based amplification, Q-Beta Replicase systems, transcription-based amplification system, and strand displacement amplification. The product of amplification is termed an amplicon.

As used herein “promoter” includes reference to a region of DNA upstream from the start of transcription and involved in recognition and binding of RNA polymerase, either I, II or III, and other proteins to initiate transcription. Promoters include necessary nucleic acid sequences near the start site of transcription, such as, in the case of a polymerase II type promoter, a TATA element or a TC-motif (144) A promoter also optionally includes distal enhancer or repressor elements, which can be located as far as several thousand base pairs from the start site of transcription.

The term “plant promoter” refers to a promoter capable of initiating transcription in plant cells.

The term “microbe promoter” refers to a promoter capable of initiating transcription in microbes.

The term “foreign promoter” refers to a promoter, other than the native, or natural, promoter, which promotes transcription of a length of DNA of viral, bacterial or eukaryotic origin, including those from microbes, plants, plant viruses, invertebrates or vertebrates.

The term “microbe” refers to any microorganism (including both eukaryotic and prokaryotic microorganisms), such as fungi, yeast, bacteria, actinomycetes, algae and protozoa, as well as other unicellular structures.

The term “plant” includes whole plants, and plant organs, and progeny of same. Plant organs comprise, e.g., shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like). The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

The term “plant storage organ” includes roots, seeds, tubers, fruits, and specialized stems.

The term “constitutive” refers to a promoter that is active under most environmental and developmental conditions, such as, for example, but not limited to, the CaMV 35S promoter and the nopaline synthase terminator.

The term “tissue-preferred promoter” refers to a promoter that is under developmental control or a promoter that preferentially initiates transcription in certain tissues.

The term “tissue-specific promoter” refers to a promoter that initiates transcription only in certain tissues.

The term “cell-type specific promoter” refers to a promoter that primarily initiates transcription only in certain cell types in one or more organs.

The term “inducible promoter” refers to a promoter that is under environmental control.

The terms “encoding” and “coding” refer to the process by which a polynucleotide, through the mechanisms of transcription and translation, provides the information to a cell from which a series of amino acids can be assembled into a specific amino acid sequence to produce a functional polypeptide, such as, for example, an active enzyme or ligand binding protein.

The terms “polypeptide,” “peptide,” “protein” and “gene product” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers Amino acids may be referred to by their commonly known three-letter or one-letter symbols Amino acid sequences are written left to right in amino to carboxy orientation, respectively. Numeric ranges are inclusive of the numbers defining the range.

The terms “residue,” “amino acid residue,” and “amino acid” are used interchangeably herein to refer to an amino acid that is incorporated into a protein, polypeptide, or peptide. The amino acid may be a naturally occurring amino acid and may encompass known analogs of natural amino acids that can function in a similar manner as the naturally occurring amino acids.

The term “3-sulfino-L-alanine” is another name for cysteine sulfinic acid, cysteine sulfinate, L-cysteinesulfinic acid, 3-sulfinoalanine, 3-sulfino-alanine, L-cysteine sulfinic acid, L-cysteine sulfinic acid, cysteine hydrogen sulfite ester or alanine 3-sulfinic acid

The terms “sulfinoalanine decarboxylase” and “SAD” refer to the protein (4.1.1.29) that catalyzes the following reaction:

3-sulfino-L-alanine=hypotaurine+CO₂

SAD is another name for cysteine-sulfinate decarboxylase, L-cysteine sulfinic acid decarboxylase, cysteine-sulfinate decarboxylase, CADCase/CSADCase, CSAD, cysteic decarboxylase, cysteine sulfinic acid decarboxylase, cysteine sulfinate decarboxylase, sulfoalanine decarboxylase, sulphinoalanine decarboxylase, and 3-sulfino-L-alanine carboxy-lyase.

Other names for hypotaurine are 2-aminoethane sulfinate, 2-aminoethylsulfinic acid, and 2-aminoethanesulfinic acid

Other names for taurine are 2-aminoethane sulfonic acid, aminoethanesulfonate, L-taurine, taurine ethyl ester, and taurine ketoisocaproic acid 2-aminoethane sulfinate.

The term “functional” with reference to SAD refers to peptides, proteins or enzymes that catalyze the SAD reactions.

The term “promiscuous” with regard to SAD or SAD enzyme activity refers to a carboxy-lyase reaction which cleaves carbon-carbon bonds and includes, but is not limited to, the following substrate and end-products:

Aspartate=beta-alanine+CO₂

Glutamate=4-aminobutanoate+CO₂

Cysteic acid=2-aminoethane sulfonate+CO₂

Note: other names for 4-aminobutanoate are gamma-aminobutyric acid (GABA).

The term “recombinant” includes reference to a cell or vector that has been modified by the introduction of a heterologous nucleic acid. Recombinant cells express genes that are not normally found in that cell or express native genes that are otherwise abnormally expressed, underexpressed, or not expressed at all as a result of deliberate human intervention, or expression of the native gene may have reduced or eliminated as a result of deliberate human intervention.

The term “recombinant expression cassette” refers to a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “transgenic plant” includes reference to a plant, which comprises within its genome a heterologous polynucleotide. Generally, the heterologous polynucleotide is integrated within the genome such that the polynucleotide is passed on to successive generations. The heterologous polynucleotide may be integrated into the genome alone or as part of a recombinant expression cassette. “Transgenic” is also used to include any cell, cell line, callus, tissue, plant part or plant, the genotype of which has been altered by the presence of heterologous nucleic acid including those transgenic plants altered or created by sexual crosses or asexual propagation from the initial transgenic plant. The term “transgenic” does not encompass the alteration of the genome by conventional plant breeding methods or by naturally occurring events such as random cross-fertilization, non-recombinant viral infection, non-recombinant bacterial transformation, non-recombinant transposition, or spontaneous mutation.

The term “vector” includes reference to a nucleic acid used in transfection or transformation of a host cell and into which can be inserted a polynucleotide.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity, and most preferably 100% sequence identity (i.e., complementary) with each other.

The terms “stringent conditions” and “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt solution. Low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Moderate stringency conditions include hybridization in 40 to 45% formamide, 1 M NaCl, 1% SDS at 37° C., and a wash in 0.5× to 1×SSC at 55 to 60° C. High stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated (145), where the Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill in the art will understand that variations in the stringency of hybridization and/or wash solutions are inherently described. An extensive guide to the hybridization of nucleic acids is found in the scientific literature (82, 146). Unless otherwise stated, in the present application high stringency is defined as hybridization in 4×SSC, 5×Denhardt solution (5 g Ficoll, 5 g polyvinypyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C.

The following terms are used to describe the sequence relationships between two or more nucleic acids or polynucleotides or polypeptides: “reference sequence,” “comparison window,” “sequence identity,” “percentage of sequence identity,” and “substantial identity.”

The term “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

The term “comparison window” includes reference to a contiguous and specified segment of a polynucleotide sequence, where the polynucleotide sequence may be compared to a reference sequence and the portion of the polynucleotide sequence in the comparison window may comprise additions or deletions (i.e., gaps) when it is compared to the reference sequence for optimal alignment. The comparison window is usually at least 20 contiguous nucleotides in length, and optionally can be 30, 40, 50, 100 or longer. Those of ordinary skill in the art understand that the inclusion of gaps in a polynucleotide sequence alignment introduces a gap penalty, and it is subtracted from the number of matches.

Methods of alignment of nucleotide and amino acid sequences for comparison are well known to those of ordinary skill in the art. The local homology algorithm, BESTFIT, (147) can perform an optimal alignment of sequences for comparison using a homology alignment algorithm called GAP (148), search for similarity using Tfasta and Fasta (149), by computerized implementations of these algorithms widely available on-line or from various vendors (Intelligenetics, Genetics Computer Group). CLUSTAL allows for the alignment of multiple sequences (150-152) and program PileUp can be used for optimal global alignment of multiple sequences (153). The BLAST family of programs can be used for nucleotide or protein database similarity searches. BLASTN searches a nucleotide database using a nucleotide query. BLASTP searches a protein database using a protein query. BLASTX searches a protein database using a translated nucleotide query that is derived from a six-frame translation of the nucleotide query sequence (both strands). TBLASTN searches a translated nucleotide database using a protein query that is derived by reverse-translation. TBLASTX search a translated nucleotide database using a translated nucleotide query.

GAP (148) maximizes the number of matches and minimizes the number of gaps in an alignment of two complete sequences. GAP considers all possible alignments and gap positions and creates the alignment with the largest number of matched bases and the fewest gaps. It also calculates a gap penalty and a gap extension penalty in units of matched bases. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (154).

Unless otherwise stated, sequence identity or similarity values refer to the value obtained using the BLAST 2.0 suite of programs using default parameters (155). As those of ordinary skill in the art understand that BLAST searches assume that proteins can be modeled as random sequences and that proteins comprise regions of nonrandom sequences, short repeats, or enriched for one or more amino acid residues, called low-complexity regions. These low-complexity regions may be aligned between unrelated proteins even though other regions of the protein are entirely dissimilar. Those of ordinary skill in the art can use low-complexity filter programs to reduce number of low-complexity regions that are aligned in a search. These filter programs include, but are not limited to, the SEG (156, 157) and XNU (158).

The terms “sequence identity” and “identity” are used in the context of two nucleic acid or polypeptide sequences and include reference to the residues in the two sequences, which are the same when aligned for maximum correspondence over a specified comparison window. When the percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conserved substitutions, the percent sequence identity may be adjusted upwards to correct for the conserved nature of the substitution. Sequences, which differ by such conservative substitutions, are said to have “sequence similarity” or “similarity.” Scoring for a conservative substitution allows for a partial rather than a full mismatch (159), thereby increasing the percentage sequence similarity.

The term “percentage of sequence identity” means the value determined by comparing two optimally aligned sequences over a comparison window, wherein the portion of the polynucleotide sequence in the comparison window may comprise gaps (additions or deletions) when compared to the reference sequence for optimal alignment. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90%, and most preferably at least 95%, compared to a reference sequence using one of the alignment programs described using standard parameters. One of ordinary skill in the art will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 50-100%. Another indication that nucleotide sequences are substantially identical is if two molecules hybridize to each low stringency conditions, moderate stringency conditions or high stringency conditions. Yet another indication that two nucleic acid sequences are substantially identical is if the two polypeptides immunologically cross-react with the same antibody in a western blot, immunoblot or ELISA assay.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm (148). Thus, a peptide is substantially identical to a second peptide, for example, where the two peptides differ only by a conserved substitution. Another indication that amino acid sequences are substantially identical is if two polypeptides immunologically cross-react with the same antibody in a western blot, immunoblot or ELISA assay. In addition, a peptide can be substantially identical to a second peptide when they differ by a non-conservative change if the epitope that the antibody recognizes is substantially identical.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Russell, 1984, Molecular biology of plants: a laboratory course manual (Cold Spring Harbor Laboratory Press, Cold Spring Harbor, N.Y.); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, 1988, Antibodies, (Cold Spring Harbor Laboratory Press, Cold Spring Harbor, N.Y.); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Fire et al., RNA Interference Technology: From Basic Science to Drug Development, Cambridge University Press, Cambridge, 2005; Schepers, RNA Interference in Practice, Wiley—VCH, 2005; Engelke, RNA Interference (RNAi): The Nuts & Bolts of siRNA Technology, DNA Press, 2003; Gott, RNA Interference, Editing, and Modification: Methods and Protocols (Methods in Molecular Biology), Human Press, Totowa, N.J., 2004; Sohail, Gene Silencing by RNA Interference: Technology and Application, CRC, 2004.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The present invention is described by reference to the following Examples, which are offered by way of illustration and are not intended to limit the invention in any manner. Standard techniques well known in the art or the techniques specifically described below were utilized.

### Example 1

### Development of a Transgenic Plant that Constitutively Expresses (AtPHYB Promoter) SAD Using Fusion PCR

Step 1: Make a DNA construct that contains an AtPHYB (Locus ID# At2g18790) promoter with a SAD gene and a NOS terminator in the following manner:

Step 1a: Use PCR to amplify the AtPHYB promoter (−1960 to −1 bps) with a short overlap for the 5′ end of SAD at the 3′ end of the promoter using 500 ng of genomic DNA isolated from an Arabidopsis thaliana Col-0. Add 300 nM of the following primers: 5′AtPHYB (SEQ ID NO:14) and AtPHYBSAD (SEQ ID NO:15). Run the fusion PCR as described in Szewczyk et al. (40).

Step 1b: Use PCR to amplify the SAD gene from 500 ng of cDNA from a zebrafish (Danio rerio) cDNA library. Add 300 nM of the following primers: 5′ SAD (SEQ ID NO:16) and 3′SAD (SEQ ID NO:17). Run the fusion PCR as described in Szewczyk et al. (40).

Step 1c: Use PCR to amplify the NOS terminator with a short overlap for the 3′ end of SAD at the 5′ end of the terminator using 500 ng of pPV1. Add 300 nM of the following primers SADNOS (SEQ ID NO:18) and 3′NOS (SEQ ID NO:19). Run the fusion PCR as described in Szewczyk et al. (40).

Step 1d: Combine the PCR fragments (Example 1: 1a, 1b, and 1c) and 300 nM of the following primers IP2 (SEQ ID NO:20) and IPS (SEQ ID NO:21). Run the fusion PCR as described in Szewczyk et al. (40). Clone into pCR4.0-TOPO as described by the manufacturer (Invitrogen).

Step 2: Transform E. coli, select for antibiotic resistance, conduct PCR identification of cloned DNA constructs in transformants, purify the plasmid that contains the DNA construct to confirm its identity and the fidelity of the sequence. Digest the plasmid with XbaI and SalI, isolate DNA fragment and ligate into the vector pCAMBIA2300 that has been predigested with XbaI and SalI.

Step 3: Transform the DNA construct into Agrobacterium tumefaciens, select for antibiotic resistance and confirm the presence of the DNA construct.

Step 4: Transform the construct into Arabidopsis thaliana, select for antibiotic resistance, select for homozygote plants and confirm the presence of the DNA constructs.

### Example 2

### Development of a Transgenic Plant that Non-Constitutively Expresses (AtGAD1 Promoter) SAD Using Chemical Synthesis

Step 1: Use chemical synthesis to make a DNA construct that contains the AtGAD1 (Locus ID# At5g17330) promoter (−1732 to −1 bps) linked to a gene for SAD (SEQ ID NO:1, SEQ ID NO:2, SEQ ID NO:3, SEQ ID NO:4, SEQ ID NO:5, SEQ ID NO:6 or SEQ ID NO:7) and a terminator NOS (from bp 2756 to bp 3033 of locus AF502128). Clone the DNA construct into a binary vector, such as pCambia1300, pCambia2300 or pCambia3200.

Step 2: Transform the DNA construct into Agrobacterium tumefaciens, select for antibiotic resistance and confirm the presence of the DNA construct.

Step 3: Transform a plant (Arabidopsis, soybean, corn, camelina, canola, rice, cotton, wheat, sugarbeet, sugarcane, or sorghum) and select for transgenic plants. Confirm the presence of the DNA constructs in the transgenic plants.

### Example 3

### Development of a Transgenic Plant that Non-Constitutively Expresses (AtGAD2 Promoter) SAD Using Chemical Synthesis

Step 1: Use chemical synthesis to make a DNA construct that contains the AtGAD2 (Locus ID# At1g65960) promoter (−1714 to −1 bps) linked to a gene for SAD (SEQ ID NO:1, SEQ ID NO:2, SEQ ID NO:3, SEQ ID NO:4, SEQ ID NO:5, SEQ ID NO:6 or SEQ ID NO:7) and a terminator NOS (from bp 2756 to bp 3033 of locus AF502128). Clone the DNA construct into a binary vector, such as pCambia1300, pCambia2300 or pCambia3200.

Step 2: Transform the DNA construct into Agrobacterium tumefaciens, select for antibiotic resistance and confirm the presence of the DNA construct.

Step 3: Transform a plant (Arabidopsis, soybean, corn, camelina, canola, rice, cotton, wheat, sugarbeet, sugarcane, or sorghum) and select for transgenic plants. Confirm the presence of the DNA constructs in the transgenic plants.

### Example 4

### Development of a Transgenic Plant that that Non-Constitutively Expresses (AtGLR1.1 Promoter) SAD Using Chemical Synthesis

Step 1: Use chemical synthesis to make a DNA construct that contains the AtGLR1.1 (Locus ID # At3g04110) promoter (−1400 to −1 bps) linked to a gene for SAD (SEQ ID NO:1, SEQ ID NO:2, SEQ ID NO:3, SEQ ID NO:4, SEQ ID NO:5, SEQ ID NO:6 or SEQ ID NO:7) and a terminator NOS (from bp 2756 to bp 3033 of locus AF502128). Clone the DNA construct into a binary vector, such as pCambia1300, pCambia2300 or pCambia3200.

Step 2: Transform the DNA construct into Agrobacterium tumefaciens, select for antibiotic resistance and confirm the presence of the DNA construct.

Step 3: Transform a plant (Arabidopsis, soybean, corn, camelina, canola, rice, cotton, wheat, sugarbeet, sugarcane, or sorghum) and select for transgenic plants. Confirm the presence of the DNA constructs in the transgenic plants.

### Example 5

### Development of a Transgenic Plant that that Non-Constitutively Expresses (AtSULTR1;3 Promoter) SAD Using Chemical Synthesis

Step 1: Use chemical synthesis to make a DNA construct that contains the AtSULTR1;3 (Locus ID # At1g22150) promoter (−2406 to −1 bps) linked to a gene for SAD (SEQ ID NO:1, SEQ ID NO:2, SEQ ID NO:3, SEQ ID NO:4, SEQ ID NO:5, SEQ ID NO:6 or SEQ ID NO:7) and a terminator NOS (from bp 2756 to bp 3033 of locus AF502128). Clone the DNA construct into a binary vector, such as pCambia1300, pCambia2300 or pCambia3200.

Step 2: Transform the DNA construct into Agrobacterium tumefaciens, select for antibiotic resistance and confirm the presence of the DNA construct.

Step 3: Transform a plant (Arabidopsis, soybean, corn, camelina, canola, rice, cotton, wheat, sugarbeet, sugarcane, or sorghum) and select for transgenic plants. Confirm the presence of the DNA constructs in the transgenic plants.

### BIBLIOGRAPHY

1. Sekowska, A, Kung, H F & Danchin, A 2000. Sulphur metabolism in Escherichia coli and related bacteria: facts and fictions. Journal of Molecular Microbiology and Biotechnology, 2: 145-177.
2. Jacobsen, J G, Thomas, L L & Smith Jr., L H 1964. Properties and distribution of mammalian L-cysteine sulfinate carboxylyase. Biochim Biophys Acta, 85: 103-116.
3. Guion-Rain, M C, Portemer, C & Chatagner, F 1975. Rat liver cysteine sulfinate decarboxylase: purification, new appraisal of the molecular weight and determination of catalytic properties. Biochim Biophys Acta, 384: 265-276.
4. Urban, P F & Reichert, P 1982. Purification and properties of specific cysteine sulphinate decarboxylase (EC 4.1.1.29) from rat brain. Biochem Soc Trans, 10: 59-60.
5. Griffith, O W 1983. Cysteinesulfinate metabolism. Altered partitioning between transamination and decarboxylation following administration of beta-methyleneaspartate. J Biol Chem, 258: 1591-1598.
6. Weinstein, C L & Griffith, O W 1987. Multiple forms of rat liver cysteinesulfinate decarboxylase. J Biol Chem, 262: 7254-7263.
7. Fouad, W M & Rathinasabapathi, B 2006. Expression of bacterial L-aspartate-alpha-decarboxylase in tobacco increases beta-alanine and pantothenate levels and improves thermotolerance Plant Mol Biol, 60: 495-505.
8. Fouad, W M & Altpeter, F 2009. Transplastomic expression of bacterial L-aspartate-alpha-decarboxylase enhances photosynthesis and biomass production in response to high temperature stress. Transgenic Research, 18: 707-718.
9. Kathiresan, A, Miranda, J, Chinnappa, C C et al., 1998. g-aminobutyric acid promotes stem elongation in Stellaria longipes: the role of ethylene. Plant Growth Reg, 26: 131-137.
10. Kinnersley, A M & Lin, F 2000. Receptor modifiers indicate that GABA is a potential modulator of ion transport in plants. Plant Growth Reg, 9: 137-146.
11. Guo, P 2009. Differentially expressed genes between drought-tolerant and drought-sensitive barley genotypes in response to drought stress during the reproductive stage. J Exp Bot, 60: 3531-3544.
12. Bolarin, M C, Santa-Cruz, A, Cayvela, E et al., 1995. Short-term solute changes in leaves and roots of cultivated and wild tomato seedlings under salinity. J Plant Physiol, 147: 463-468.
13. Aurisano, N, Bertani, A & Reggiani, R 1995. Anaerobic accumulation of 4-aminobutyrate in rice seedlings; causes and significance. Phytochemistry, 38: 1147-1150.
14. Wahid 2007. Heat tolerance in plants: An overview. Environ Exp Bot, 61: 199-223.
15. Jaffe, M J & Biro, R 1979. Thigmomorphogenesis, the effect of mechanical perturbation on the growth of plants, with special reference to anatomical changes, the role of ethylene and interaction with other environmental stresses. In Mussell H & RC Staples, editors. Stress physiology in crop plants 25-69. New York: John Wiley & Sons.
16. Kinnersley, A M & Turano, F J 2000. Gamma-aminobutyric acid (GABA) and plant responses to stress. Crit. Rev Plant Sci, 19: 479-509.
17. Allan 2006. Fluctuations of (γ-aminobutyrate, (γ-hydroxybutyrate and related amino acids in Arabidopsis leaves as a function of the light-dark cycle, leaf age and N stress. Can J Bot, 84: 1339-1346.
18. MacGregor, K B, Shelp, B J, Peiris, S et al., 2003. Overexpression of glutamate decarboxylase in transgenic tobacco plants deters feeding by phytophagous insect larvae. J Chem Ecol, 29: 2177-82.
19. Bown, A W 2006. Gamma-aminobutyrate: defense against invertebrate pests? Trends Plant Sci, 11: 424-427.
20. Bown, A W & Shelp, B J 1997. The metabolism and functions of [gamma]-aminobutyric acid. Plant Physiol, 115: 1-5.
21. Selman, I W & Cooper, P 1978. Changes in the free amino compounds in young tomato plants in light and darkness with particular reference to g-aminobutyric acid. Ann Bot, 42: 627-636.
22. Beuve, N, Rispail, N, Laine, P et al., 2004. Putative role of g-aminobutyric acid (GABA) as a long-distance signal in up-regulation of nitrate uptake in Brassica napus L. Plant Cell Environ, 27: 1035-1046.
23. Satyanarayan, V & Nair, P M 1990. Metabolism, enzymology and possible roles of 4-amino butyrate in higher plants. Phytochemistry, 29: 367-375.
24. Roberts, M R 2007. Does GABA act as a signal in plants? Hints from molecular studies. Plant Signaling & Behavior, 2: 408-409.
25. Schweigen, R G 1967. Low-molecular-weight compounds in Macrocystis pyrifera, a marine algae. Arch Biochem Biophys, 118: 383-387.
26. Huxtable, R J 1992. Physiological actions of taurine. Physiol Rev, 72: 101-163.
27. Kataoka, H & Ohnishi, N 1986. Occurrence of taurine in plants. Agric Biol Chem, 50: 1887-1888.
28. Suzuki, A, Kajita, T & Furushima, M 1989. U.S. Pat. No. 4,877,447.
29. Murray, E E, Lotzer, J & Eberle, M 1989. Codon usage in plant genes. Nucleic Acids Research, 17: 477-498.
30. Ingelbrecht, I L, Herman, L M, Dekeyser, R A et al., 1989. Different 3′ end regions strongly influence the level of gene expression in plant cells. Plant Cell, 1: 671-680.
31. An, G, Mitra, A, Choi, H K et al., 1989. Functional analysis of the 3′ control region of the potato wound-inducible proteinase inhibitor II gene. Plant Cell, 1: 115-122.
32. Langenheim, J H & Thimann, K V 1982. Botany: Plant Biology and its Relation to Human Affairs. New York: John Wiley & Sons Inc.
33. Vasil, I K 1984. Cell Culture and Somatic Cell Genetics of Plants: Laboraory Procedures and Their Applications. Orlando: Academic Press.
34. Stanier, R, Ingrahm, J, Wheelis, M et al., 1986. The Microbial World. New Jersey: Prentice-Hall.
35. Dhringra, O D & Sinclair, J B 1985. Basic plant pathology methods. Boca Raton, Fla.: CRC Press.
36. Maniatis, T, Fritsch, E F & Sambrook, J 1985. Molecular Cloning: A Laboratory Manual: DNA Cloning. New York: Cold Spring Harbor.
37. Gait 1984. Oligonucleotide Synthesis-A Practical Approach. Washington, D.C.: IRL Press.
38. Hames, D D & Higgins, S J 1984. Nucleic Acid Hybridization: A Practical Approach. Washington D.C.: IRL Press.
39. Watson, J D, Gilman, M, Witowski, J et al., 1992. Recombinant DNA. New York: Scientific American Books.
40. Szewczyk, E, Nayak, T, Oakley, C E et al., 2006. Fusion PCR and gene targeting in Aspergillus nidulans. Nat Protoc, 1: 3111-3121.
41. Ho, S N, Hunt, H D, Horton, R M et al., 1989. Site-directed mutagenesis by overlap extension using the polymerase chain reaction. Gene, 77: 51-59.
42. Fuhrmann, M, Oertel, W & Hegemann, P 1999. A synthetic gene coding for the green fluorescent protein (GFP) is a versatile reporter in Chlamydomonas reinhardtii. Plant J, 19: 353-361.
43. Mandecki, W & Bolling, T J 1988. FokI method of gene synthesis. Gene, 68: 101-107.
44. Stemmer, W P, Crameri, A., Ha, K. D., Brennan, T. M. and Heyneker, H. L. 1995. Single-step assembly of a gene and entire plasmid from large numbers of oligodeoxyribonucleotides. Gene, 164: 49-53.
45. Gao, X, Yo, P, Keith, A et al., 2003. Thermodynamically balanced inside-out (TBIO) PCR-based gene synthesis: a novel method of primer design for high-fidelity assembly of longer gene sequences. Nucleic Acids Res, 31: e143.
46. Young, L & Dong, Q 2004. Two-step total gene synthesis method. Nucleic Acids Res, 32: e59.
47. van Der Krol, A R, van Poecke, R M, Vorst, O F et al., 1999. Developmental and wound-, cold-, desiccation-, ultraviolet-B-stress-induced modulations in the expression of the petunia zinc finger transcription factor gene ZPT2-2. Plant Physiol, 121: 1153-62.
48. Shinmyo, A, Shoji, T, Bando, E et al., 1998. Metabolic engineering of cultured tobacco cells. Biotechnol Bioeng, 58: 329-32.
49. Sohal, A K, Pallas, J A & Jenkins, G I 1999. The promoter of a Brassica napus lipid transfer protein gene is active in a range of tissues and stimulated by light and viral infection in transgenic Arabidopsis. Plant Mol Biol, 41: 75-87.
50. Eulgem, T, Rushton, P J, Schmelzer, E et al., 1999. Early nuclear events in plant defense signalling: rapid gene activation by WRKY transcription factors. EMBO (Eur Mol Biol Organ) J, 18: 4689-99.
51. Cormack, R S, Eulgem, T, Rushton, P J et al., 2002. Leucine zipper-containing WRKY proteins widen the spectrum of immediate early elicitor-induced WRKY transcription factors in parsley. Biochim Biophys Acta, 1576: 92-100.
52. Lebel, E, Heifetz, P, Thorne, L et al., 1998. Functional analysis of regulatory sequences controlling PR-1 gene expression in Arabidopsis. Plant J, 16: 223-33.
53. Ngai, N, Tsai, F Y & Coruzzi, G 1997. Light-induced transcriptional repression of the pea AS1 gene: identification of cis-elements and transfactors. Plant J, 12: 1021-34.
54. Kucho, K, Ohyama, K & Fukuzawa, H 1999. CO(2)-responsive transcriptional regulation of CAH1 encoding carbonic anhydrase is mediated by enhancer and silencer regions in Chlamydomonas reinhardtii. Plant Physiol, 121: 1329-38.
55. Kucho, K, Yoshioka, S, Taniguchi, F et al., 2003. Cis-acting elements and DNA-binding proteins involved in CO2-responsive transcriptional activation of Cah1 encoding a periplasmic carbonic anhydrase in Chlamydomonas reinhardtii. Plant Physiol, 133: 783-93.
56. Chen, W & Singh, K B 1999. The auxin, hydrogen peroxide and salicylic acid induced expression of the Arabidopsis GST6 promoter is mediated in part by an ocs element. Plant J, 19: 667-77.
57. Chen, W, Chao, G & Singh, K B 1996. The promoter of a H2O2-inducible, Arabidopsis glutathione S-transferase gene contains closely linked OBF- and OBP1-binding sites. Plant J, 10: 955-66.
58. Lu, C A, Lim, E K & Yu, S M 1998. Sugar response sequence in the promoter of a rice alpha-amylase gene serves as a transcriptional enhancer. J Biol Chem, 273: 10120-31.
59. Leubner-Metzger, G, Petruzzelli, L, Waldvogel, R et al., 1998. Ethylene-responsive element binding protein (EREBP) expression and the transcriptional regulation of class I beta-1,3-glucanase during tobacco seed germination. Plant Mol Biol, 38: 785-95.
60. de Framond, A J 1991. A metallothionein-like gene from maize (Zea mays). Cloning and characterization. FEBS Lett, 290: 103-6.
61. Hudspeth, R L, Hobbs, S L, Anderson, D M et al., 1996. Characterization and expression of metallothionein-like genes in cotton. Plant Mol Biol, 31: 701-5.
62. Herrera-Estrella, L, Depicker, A, van Montagu, M et al., 1983. Expression of chimaeric genes transferred into plant cells using a Ti-plasmid-derived vector. Nature, 303: 209-213.
63. Pathirana, M S, Samac, D A, Roeven, R et al., 1997. Analyses of phosphoenolpyruvate carboxylase gene structure and expression in alfalfa nodules. Plant J, 12: 293-304.
64. Yang, S H, Yu, H & Goh, C J 2002. Isolation and characterization of the orchid cytokinin oxidase DSCKX1 promoter. J Exp Bot, 53: 1899-1907.
65. Moon, H & Callahan, A M 2004. Developmental regulation of peach ACC oxidase promoter-GUS fusions in transgenic tomato fruits. J Exp Bot, 55: 1519-1528.
66. Avsian-Kretchmer, O, Gueta-Dahan, Y, Lev-Yadun, S et al., 2004. The salt-stress signal transduction pathway that activates the gpx1 promoter is mediated by intracellular H2O2, different from the pathway induced by extracellular H2O2. Plant Physiol, 135: 1685-96.
67. Wu, A-M, Lv, S-Y & Liu, J-Y 2007. Functional analysis of a cotton glucuronosyltransferase promoter in transgenic tobaccos. Cell Res, 17: 174-183.
68. Shelp, B J, Bown, A W & McLean, M D 1999. Metabolism and functions of gamma-aminobutyric acid. Trends Plant Sci, 41: 446-452.
69. Baum, G, Chen, Y, Arazi, T et al., 1993. A plant glutamate decarboxylase containing a calmodulin binding domain. Cloning, sequence, and functional analysis. J Biol Chem, 268: 19610-19617.
70. Gallego, P P, Whotton, L, Picton, S et al., 1995. A role for glutamate decarboxylase during tomato ripening: the characterisation of a cDNA encoding a putative glutamate decarboxylase with a calmodulin-binding site. Plant Mol Biol, 27: 1143-1151.
71. Yun, S J & Oh, S-H 1998. Cloning and characterization of tobacco cDNA encoding calcium/calmodulin-dependent glutamate decarboxylase. Mol Cell, 8: 125-129.
72. Oh, S H, Choi, W G, Lee, I T et al., 2005. Cloning and characterization of a rice cDNA encoding glutamate decarboxylase. J Biochem Mol Biol, 38: 595-601.
73. Chiu, J C, Brenner, E D, DeSalle, R et al., 2002. Phylogenetic and expression analysis of the glutamate-receptor-like gene family in Arabidopsis thaliana. Mol Biol Evol, 19: 1066-1082.
74. Kim, S A, Kwak, J M, Jae, S K et al., 2001. Overexpression of the AtGluR2 gene encoding an Arabidopsis homolog of mammalian glutamate receptors impairs calcium utilization and sensitivity to ionic stress in transgenic plants. Plant Cell Physiol, 42: 74-84.
75. Meyerhoff, O, Muller, K, Roelfsema, M R et al., 2005. AtGLR3.4, a glutamate receptor channel-like gene is sensitive to touch and cold. Planta, 222: 418-27.
76. Maruyama-Nakashita, A, Nakamura, Y, Yamaya, T et al., 2004. Regulation of high-affinity sulphate transporters in plants towards systematic analysis of sulphur signalling and regulation. J Exp Bot, 55: 1843-1849.
77. Yoshimoto, N, Inoue, E, Saito, K et al., 2003. Phloem-localizing sulfate transporter, Sultr1;3, mediates re-distribution of sulfur from source to sink organs in Arabidopsis. Plant Physiol, 131: 1511-7.
78. Awazuhara, M, Fujiwara, T, Hayashi, H et al., 2005. The function of SULTR2;1 sulfate transporter during seed development in Arabidopsis thaliana. Physiol Plant, 125: 95-105.
79. Kataoka, T, Hayashi, N, Yamaya, T et al., 2004. Root-to-shoot transport of sulfate in Arabidopsis: Evidence for the role of SULTR3;5 as a component of low-affinity sulfate transport system in the root vasculature. Plant Physiol, 136: 4198-4204.
80. An, G, Watson, B D, Stachel, S et al., 1985. New cloning vehicles for transformation of higher plants. EMBO (Eur Mol Biol Organ) J, 4: 277-284.
81. Gruber, M Y & Cosby, W L 1993. Vectors for plant transformation. In Glick B R & J E Thompson, editors. Methods in Plant Molecular Biology and Biotechnology 89-119. Baco Raton, Fla.: CRC Press.
82. Ausubel, F M, Brent, R, Kingston, R E et al., 1995. Current Protocols in Molecular Biology. New York: Greene Publishing and Wiley-Interscience.
83. Ohme-Takagi, M, Taylor, C B, Newman, T C et al., 1993. The effect of sequences with high AU content on mRNA stability in tobacco. Proc Natl Acad Sci USA, 90: 11811-5.
84. Newman, T C, Ohme-Takagi, M, Taylor, C B et al., 1993. DST sequences, highly conserved among plant SAUR genes, target reporter transcripts for rapid decay in tobacco. Plant Cell, 5: 701-14.
85. von Heijne, G 1986. Mitochondrial targeting sequences may form amphiphilic helices. EMBO (Eur Mol Biol Organ) J, 5: 1335-1342.
86. Swinkels, B W, Gould, S J, Bodnar, A G et al., 1991. A novel, cleavable peroxisomal targeting signal at the amino-terminus of the rat 3-ketoacyl-CoA thiolase. EMBO (Eur Mol Biol Organ) J, 10: 3255-62.
87. Rusch, S L & Kendall, D A 1995. Protein transport via amino-terminal targeting sequences: Common themes in diverse systems. Mol Membr Biol, 12: 295-307.
88. Soll, J & Tien, R 1998. Protein translocation into and across the chloroplastic envelope membranes. Plant Mol Biol, 38: 191-207.
89. Gould, S J, Keller, G A & Subramani, S 1988. Identification of peroxisomal targeting signals located at the carboxy terminus of four peroxisomal proteins. J Cell Biol, 107: 897-905.
90. Gould, S J, Keller, G A, Hosken, N et al., 1989. A conserved tripeptide sorts proteins to peroxisomes. J Cell Biol, 108: 1657-64.
91. McCammon, M T, McNew, J A, Willy, P J et al., 1994. An internal region of the peroxisomal membrane protein PMP47 is essential for sorting to peroxisomes. J Cell Biol, 124: 915-25.
92. Cokol, M, Nair, R & Rost, B 2000. Finding nuclear localization signals. EMBO Rep, 1: 411-5.
93. Helenius, A & Aebi, M 2001. Intracellular functions of N-linked glycans. Science, 291: 2364-9.
94. Emanuelsson, O, Brunak, S, von Heijne, G et al., 2007. Locating proteins in the cell using TargetP, SignalP and related tools. Nat Protoc, 2: 953-971.
95. Emanuelsson, O, Nielsen, H, Brunak, S et al., 2000. Predicting subcellular localization of proteins based on their N-terminal amino acid sequence. J Mol Biol, 300: 1005-1016.
96. Bannai, H, Tamada, Y, Maruyama, O et al., 2002. Extensive feature detection of N-terminal protein sorting signals. Bioinformatics, 18: 298-305.
97. Bendtsen, J D, Nielsen, H, von Heijne, G et al., 2004. Improved prediction of signal peptides: SignalP 3.0. J Mol Biol, 340: 783-95.
98. Hiller, K, Grote, A, Scheer, M et al., 2004. PrediSi: prediction of signal peptides and their cleavage positions. Nucleic Acids Res, 32: W375-9.
99. Bhasin, M & Raghava, G P 2004. ESLpred: SVM-based method for subcellular localization of eukaryotic proteins using dipeptide composition and PSI-BLAST. Nucleic Acids Res, 32: W414-9.
100. Garg, A, Bhasin, M & Raghava, G P 2005. Support vector machine-based method for subcellular localization of human proteins using amino acid compositions, their order, and similarity search. J Biol Chem, 280: 14427-32.
101. Bhasin, M, Garg, A & Raghava, G P 2005. PSLpred: prediction of subcellular localization of bacterial proteins. Bioinformatics, 21: 2522-4.
102. Hoglund, A, Donnes, P, Blum, T et al., 2006. MultiLoc: prediction of protein subcellular localization using N-terminal targeting sequences, sequence motifs and amino acid composition. Bioinformatics, 22: 1158-65.
103. Shatkay, H, Hoglund, A, Brady, S et al., 2007. SherLoc: high-accuracy prediction of protein subcellular localization by integrating text and protein sequence data. Bioinformatics, 23: 1410-7.
104. Emanuelsson, O, Nielsen, H & von Heijne, G 1999. ChloroP, a neural network-based method for predicting chloroplast transit peptides and their cleavage sites. Protein Sci, 8: 978-984.
105. Claros, M G & Vincens, P 1996. Computational method to predict mitochondrially imported proteins and their targeting sequences. Eur J Biochem, 241: 779-86.
106. Small, I, Peeters, N, Legeai, F et al., 2004. Predotar: A tool for rapidly screening proteomes for N-terminal targeting sequences. Proteomics, 4: 1581-1590.
107. Kelley, L A, MacCallum, R M & Sternberg, M J 2000. Enhanced genome annotation using structural profiles in the program 3D-PSSM. J Mol Biol, 299: 499-520.
108. Shahin, E A 1985. Totipotency of tomato protoplasts. Theor Appl Genet, 69: 235-240.
109. Fromm, M, Taylor, L P & V., W 1985. Expression of genes transferred into monocot and dicot plant cells by electroporation. Proc Natl Acad Sci USA, 82: 5824-5828.
110. Fromm, M E, Taylor, L P & Walbot, V 1986. Stable transformation of maize after gene transfer by electroporation. Nature, 319: 791-3.
111. Riggs, C D & Bates, G W 1986. Stable transformation of tobacco by electroporation: evidence for plasmid concatenation. Proc Natl Acad Sci USA, 83: 5602-5606.
112. D'Halluin, K, Bonne, E, Bossut, M et al., 1992. Transgenic maize plants by tissue electroporation. Plant Cell, 4: 1495-1505.
113. Laursen, C M, Krzyzek, R A, Flick, C E et al., 1994. Production of fertile transgenic maize by electroporation of suspension culture cells Plant Mol Biol, 24: 51-61
114. Crossway, A, Oakes, J V, Irvine, J M et al., 1986. Integration of foreign DNA following microinjection of tobacco mesophyllprotoplasts. Mol Gen Genet, 202: 179-185.
115. Griesbach, R J 1983. Protoplast microinjection. Plant Mol Biol Report, 1: 32-37.
116. Sporlein, B & Koop, H-U 1991. Lipofectin: direct gene transfer to higher plants using cationic liposomes. Theor Appl Genet, 83: 1-5.
117. Ohgawara, T, Uchimiya, H & Harada, H 1983. Uptake of liposome-encapsulated plasmid DNA by plant protoplasts and molecular fate of foreign DNA Protoplasma, 116: 145-148.
118. Deshayes, A, Herrera-Estrella, L & Caboche, M 1985. Liposome-mediated transformation of tobacco mesophyllprotoplasts by an Escherichia coli plasmid. EMBO (Eur Mol Biol Organ) J, 4: 2731-7.
119. Christou, P, Murphy, J E & Swain, W F 1987. Stable transformation of soybean by electroporation and root formation from transformed callus. Proc Natl Acad Sci USA, 84: 3962-3966.
120. Horsch, R B, Fry, J E, Hoffmann, N L et al., 1985. A Simple and General Method for Transferring Genes into Plants. Science, 227: 1229-1231.
121. Paszkowski, J, Shillito, R D, Saul, M et al., 1984. Direct gene transfer to plants. Embo J, 3: 2717-2722.
122. Hooykaas-Van Slogteren, G M, Hooykaas, P J & Schilperoort, R A 1984. Expression of Ti plasmid genes in monocotyledonous plants infected with Agrobacterium tumefaciens. Nature, 311: 763-764.
123. Rogers, S G, Horsch, R. B., and Fraley, R. T. 1986. Gene transfer in plants: Production of transformed plants using Ti-plasmid vectors. 1986. Gene transfer in plants: Production of transformed plants using Ti-plasmid vectors. Methods Enzymol, 118: 627-640.
124. Bevan, M W & Chilton, M-D 1982. T-DNA of the Agrobacterium Ti and Ri plasmids. Annu Rev Genet, 16: 357-384.
125. Klein, T M, Fromm, M, Weissinger, A et al., 1988. Transfer of foreign genes into intact maize cells with high-velocity microprojectiles. Proc Natl Acad Sci USA, 85: 4305-4309.
126. Klein, T M, Gradziel, T, Fromm, M E et al., 1988. Factors influencing gene delivery into Zea mays cells by high-velocity microprojectiles. Biotechnology, 6: 559-563.
127. McCabe, D E, Swain, W F, Martinell, B J et al., 1988. Stable transformation of soybean (Glycine max) by particle acceleration. Biotechnology, 6: 923-926.
128. Sanford, J C, Smith, F D & Rushell, J A 1993. Optimizing the biolistic process for different biological application. In Wu R, editor. The Methods in Enzymology 483-509. Orlando: Academic Press.
129. Freeman, J P, Draper, J, Davey, M R et al., 1984. A Comparison of Methods for Plasmid Delivery into Plant Protoplasts. Plant Cell Physiol, 25: 1353-1365.
130. Frame, B R, Drayton, P R, Bagnall, S V et al., 1994. Production of fertile transgenic maize plants by silicon carbide whisker-mediated transformation. Plant J, 6: 941-948.
131. Thompson, J A, Drayton, P, Frame, B et al., 1995. Maize transformation utilizing silicon carbide whiskers: a review. Euphytica, 85: 75-80.
132. Guo, Y, Liang, H & Berns, M W 1995. Laser-mediated gene transfer in rice. Physiol Plant, 93: 19-24.
133. Badr, Y A, Kereim, M A, Yehia, M A et al., 2005. Production of fertile transgenic wheat plants by laser micropuncture. Photochem Photobiol Sci, 4: 803-807.
134. Bao, S, Thrall, B D & Miller, D L 1997. Transfection of a reporter plasmid into cultured cells by sonoporation in vitro. Ultrasound in Medicine and Biology, 23: 953-959.
135. Finer, K R & Finer, J J 2000. Use of Agrobacterium expressing green fluorescent protein to evaluate colonization of sonication-assisted Agrobacterium-mediated transformation-treated soybean cotyledons. Lett Appl Microbiol, 30: 406-10.
136. Amoah, B K, Wu, H, Sparks, C et al., 2001. Factors influencing Agrobacterium-mediated transient expression of uidA in wheat inflorescence tissue. J Exp Bot, 52: 1135-42.
137. Krens, F A, Molendijk, L, Wullems, G J et al., 1982. In Vitro transformation of plant protoplasts with Ti-plasmid DNA. Nature, 296: 72-74.
138. Bechtold, N & Pelletier, G 1998. In planta Agrobacterium-mediated transformation of adult Arabidopsis thaliana plants by vacuum infiltration. Methods Mol Biol, 82: 259-66.
139. Broothaerts, W, Mitchell, H J, Weir, B et al., 2005. Gene transfer to plants by diverse species of bacteria. Nature, 433: 629-633.
140. Kirk, R E & Othmer, D F 1993. Concise Encyclopedia of Chemical Technology: John Wiley & Sons.
141. Mosbach, K, Birnbaum, S, Hardy, K et al., 1983. Formation of proinsulin by immobilized Bacillus subtilis. Nature, 302: 543-545.
142. Chan, H W & Wells, R D 1974. Structural uniqueness of lactose operator. Nature, 252: 205-209.
143. Goeddel, D V, Shephard, H M, Yelverton, E et al., 1980. Synthesis of human fibroblast interferon by E. coli Nucleic Acids Res, 8: 4057-4074.
144. Bernard, V, Brunaud, V & Lecharny, A 2010. TC-motifs at the TATA-box expected position in plant genes: a novel class of motifs involved in the transciption regulation. BMC Genomics, 11: 166.
145. Meinkoth, J & G., W 1984. Hybridization of nucleic acids immobilized on solid supports. Anal Biochem, 138: 267-284.
146. Tijssen, P 1993. Overview of principles of hybridization and the strategy of nucleic acid probe assays. Laboratory Techniques in Biochemistry and Molecular Biology—Hybridization with Nucleic Acid Probes: Part I. New York: Elsevier.
147. Smith, T F & Waterman, M S 1981. Comparison of biosequences. Adv Appl Math, 2: 482-489.
148. Needleman, S B & Wunsch, C D 1970. A general method applicable to the search for similarities in the amino acid sequence of two proteins. J Mol Biol, 48: 443-453.
149. Pearson, W R & Lipman, D J 1988. Improved tools for biological sequence comparison. Proc Natl Acad Sci USA, 85: 2444-2448.
150. Higgins, D G & Sharp, P M 1989. Fast and sensitive multiple sequence alignments on a microcomputer. Comput Appl Biosci, 5: 151-153.
151. Higgins, D G & Sharp, P M 1988. CLUSTAL: a package for performing multiple sequence alignment on a microcomputer. Gene, 73: 237-244.
152. Higgins, D G, Bleasby, A J & Fuchs, R 1992. CLUSTAL V: improved software for multiple sequence alignment. Comput Appl Biosci, 8: 189-191.
153. Feng, D F & Doolittle, R F 1987. Progressive sequence alignment as a prerequisite to correct phylogenetic trees. J Mol Evol, 25: 351-360.
154. Henikoff, S & Henikoff, J 1989 Amino acid substitution matrices from protein blocks Proc Natl Acad Sci USA, 89: 10915-10919.
155. Altschul, S F, Madden, T L, Schaffer, A A et al., 1997. Gapped BLAST and PSI-BLAST: a new generation of protein database search programs. Nucleic Acids Res, 25: 3389-3402.
156. Wootton, J C & Federhen, S 1993. Statistics of local complexity in amino acid sequences and sequence databases. Comput Chem, 17: 149-163.
157. Wootton, J C & Federhen, S 1996. Analysis of compositionally biased regions in sequence databases. Methods Enzymol, 266: 554-571.
158. Clayerie, J-M & States, D J 1993. Information enhancement methods for large scale sequence analysis. Comput Chem, 17: 191-201.
159. Myers, E W & Miller, W 1988. Optimal alignments in linear-space. Comput Appl Biol Sci, 4: 11-17.
160. McLean M. D., Yevtushenko D. P., Deschene A., et al., 2003. Overexpression of glutamate decarboxylase in transgenic tobacco plants confers resistance to the northern rootknot nematode. Mol. Breed. 11, 277-285.

1. A cell comprising an expression cassette which comprises a promoter operably linked to an exogenous polynucleotide that encodes a sulfinoalanine decarboxylase (SAD) that is operably linked to a terminator.

2. The cell of claim 1, wherein the sulfinoalanine decarboxylase is encoded by a polynucleotide comprising a nucleotide sequence selected from the group consisting of the nucleotide sequence set forth in SEQ ID NO:1, the nucleotide sequence set forth in SEQ ID NO:2, the nucleotide sequence set forth in SEQ ID NO:3, the nucleotide sequence set forth in SEQ ID NO:4, the nucleotide sequence set forth in SEQ ID NO:5, the nucleotide sequence set forth in SEQ ID NO:6, and the nucleotide sequence set forth in SEQ ID NO:7.

3. The cell of claim 1, wherein the sulfinoalanine decarboxylase comprises an amino acid sequence selected from the group consisting of the amino acid sequence set forth in SEQ ID NO:8, the amino acid sequence set forth in SEQ ID NO:9, the amino acid sequence set forth in SEQ ID NO:10, the amino acid sequence set forth in SEQ ID NO:11, the amino acid sequence set forth in SEQ ID NO:12, and the amino acid sequence set forth in SEQ ID NO:13.

4. The cell of claim 1, wherein the promoter is a constitutive promoter or a non-constitutive promoter selected from the group consisting of a tissue-preferred promoter, a tissue-specific promoter, a cell type-specific promoter, an inducible promoter, a plant glutamate decarboxylase (GAD) promoter, a plant sulphate transporter (SULTR) promoter, and a plant glutamate receptor (GLR) promoter.

5. The cell of claim 1 which is a plant cell.

6. The cell of claim 1 which is an algae.

7. The cell of claim 1 wherein the polynucleotide encoding a sulfinoalanine decarboxylase further comprises a subcellular location sequence that targets a subcellular location selected from the group consisting of an apoplast, vacuole, plastid, chloroplast, proplastid, etioplast, chromoplast, mitochondrion, peroxisome, glyoxysome, nucleus, lysosome, endomembrane system, endoplasmic reticulum, vesicle, and Golgi apparatus.

8. A plant storage organ comprising the cell of claim 5, wherein the plant storage organ is a seed, tuber, fruit, or root.

9. A seed having stably incorporated in its genome an exogenous polynucleotide encoding a sulfinoalanine decarboxylase protein.

10. The seed of claim 9 wherein the exogenous polynucleotide comprises a nucleotide sequence selected from the group consisting of the nucleotide sequence set forth in SEQ ID NO:1, the nucleotide sequence set forth in SEQ ID NO:2, the nucleotide sequence set forth in SEQ ID NO:3, the nucleotide sequence set forth in SEQ ID NO:4, the nucleotide sequence set forth in SEQ ID NO:5, the nucleotide sequence set forth in SEQ ID NO:6 and the nucleotide sequence set forth in SEQ ID NO:7.

11. The seed of claim 9, wherein the sulfinoalanine decarboxylase comprises an amino acid sequence selected from the group consisting of the amino acid sequence set forth in SEQ ID NO:8, the amino acid sequence set forth in SEQ ID NO:9, the amino acid sequence set forth in SEQ ID NO:10, the amino acid sequence set forth in SEQ ID NO:11, SEQ ID NO:12, and the amino acid sequence set forth in SEQ ID NO:13.

12. A plant grown from the seed of claim 9.

13. A plant having stably incorporated in its genome an exogenous polynucleotide encoding a sulfinoalanine decarboxylase protein.

14. The plant of claim 13, wherein the plant has increased growth, yield, or biomass, altered development, increased water-use-efficiency, increased nitrogen-use-efficiency, or increased tolerance to biotic stress (pests, pathogens, bacteria, microbes, viruses, viroids, microorganisms, invertebrates, insects, nematodes, vertebrates) or abiotic stress (osmotic stress, oxidative damage, drought, salt, cold, freezing, heat, UV light, limitations of nutrients such as nitrogen, sulfur, phosphorous or other minerals).

15. The plant of claim 13 which is selected from the group consisting of acacia, alfalfa, algae, aneth, apple, apricot, artichoke, arugula, asparagus, avocado, banana, barley, beans, beech, beet, Bermuda grass, blackberry, blueberry, Blue grass, broccoli, brussels sprouts, cabbage, camelina, canola, cantaloupe, carrot, cassava, cauliflower, celery, cherry, chicory, cilantro, citrus, clementines, coffee, corn, cotton, cucumber, duckweed, Douglas fir, eggplant, endive, escarole, eucalyptus, fennel, fescue, figs, forest trees, garlic, gourd, grape, grapefruit, honey dew, jatropha, jicama, kiwifruit, lettuce, leeks, lemon, lime, Loblolly pine, maize, mango, melon, mushroom, nectarine, nut, oat, okra, onion, orange, ornamental plants, palm, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, rye grass, seaweed, scallion, sorghum, Southern pine, soybean, spinach, squash, strawberry, sugarbeet, sugarcane, sunflower, sweet potato, sweetgum, switchgrass, tangerine, tea, tobacco, tomato, turf, turnip, a vine, watermelon, wheat, yam, and zucchini.

16. A method of producing a crop of plants having stably incorporated in their genome an exogenous polynucleotide encoding a sulfinoalanine decarboxylase protein, the method comprising growing, multiplying or breeding the seed of claim 9 to obtain a crop of plants.

17. A pharmaceutical composition comprising an extract of the plant of claim 13.

18. A nutritional supplement comprising an extract of the plant of claim 13.

19. An animal feed supplement comprising:
the plant storage organ of claim 8.

20. The plant of claim 13, wherein the exogenous polynucleotide comprises a nucleotide sequence selected from the group consisting of the nucleotide sequence set forth in SEQ ID NO:1, the nucleotide sequence set forth in SEQ ID NO:2, the nucleotide sequence set forth in SEQ ID NO:3, the nucleotide sequence set forth in SEQ ID NO:4, the nucleotide sequence set forth in SEQ ID NO:5, the nucleotide sequence set forth in SEQ ID NO:6 and the nucleotide sequence set forth in SEQ ID NO:7.

21. The plant of claim 13, wherein the sulfinoalanine decarboxylase comprises an amino acid sequence selected from the group consisting of the amino acid sequence set forth in SEQ ID NO:8, the amino acid sequence set forth in SEQ ID NO:9, the amino acid sequence set forth in SEQ ID NO:10, the amino acid sequence set forth in SEQ ID NO:11, SEQ ID NO:12, and the amino acid sequence set forth in SEQ ID NO:13.

22. A method of producing a crop of plants having stably incorporated in their genome an exogenous polynucleotide encoding a sulfinoalanine decarboxylase protein, the method comprising growing, multiplying or breeding the plant of claim 12 to obtain a crop of plants.

23. A method of producing a crop of plants having stably incorporated in their genome an exogenous polynucleotide encoding a sulfinoalanine decarboxylase protein, the method comprising growing, multiplying or breeding the plant of claim 13 to obtain a crop of plants.

24. An animal feed supplement comprising the seed of claim 9.

25. An animal feed supplement comprising the plant of claim 13.
